SEQUENCE LISTING (7 sequences)

SEQ ID NO: 1 - DNA, 452 bp, Artificial
  Synthetic primer
tacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactca

SEQ ID NO: 2 - DNA, 646 bp, Artificial
  Synthetic primer
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacgggctcgactagagtcggggcggccggccgcttcgag

SEQ ID NO: 3 - DNA, 819 bp, Artificial
  Synthetic primer
ttatgtcacaccacagaagtaaggttccttcacaaagatcccaagctgtcgatcgacatttctagaggatctcggacccggggaatccccgtcccccaacatgtccagatcgaaatcgtctagcgcgtcggcatgcgccatcgccacgtcctcgccgtctaagtggagctcgtcccccaggctgacatcggtcgggggggcggatctcggacccggggaatccccgtcccccaacatgtccagatcgaaatcgtctagcgcgtcggcatgcgccatcgccacgtcctcgccgtctaagtggagctcgtcccccaggctgacatcggtcgggggggcggatcccccgggctgcaggaattccggcgatacagtcaactgtctttgacctttgttactactctcttccgatgatgatgtcgcacttattctatgctgtctcaatgttagaggcatatcagtctccactgaagccaatctatctgtgacggcatctttattcacattatcttgtacaaataatcctgttaacaatgcttttatatcctgtaaagaatccattttcaaaatcatgtcaaggtcttctcgaggaaaaatcagtagaaatagctgttccagtctttctagccttgattccacttctgtcagatgtgccctagtcagcggagaccttttggttttgggagagtagcgacactcccagttgttcttcagacacttggcgcacttcggtttttctttggagcacttgagctttttaagtcggcaaatatcgcatgcttgttcgatagaagacagtagcttcatctttcaggaggctag

SEQ ID NO: 4 - DNA, 496 bp, Artificial
  Synthetic primer
ggccgccccacgtgcgcagcaggacgcagcgctgcctgaaactcgcgccgcgaggagagggcggggccgcggaaaggaaaggggggggctgggaggcccggagggggctgggccggggacccgggaggggtcgggacggggcggggtccgcgcggaggaggcggagctggaaggtgaaggggcaggacgggtgcccgggtccccagtccctccgccacgtggggagcgcggtcctgggcgtctgtgcccgcgaatccactgggagcccggcctggccccgacagcgcagctgctccgggcggacccgggggtctgggccgcgcttccccgcccgcgcgccgctcgcgctcccagggtgcagggacgccagcgagggccccagcggagagaggtcgaatcggcctaggctgtggggtaacccgagggaggggcctctagatataagggcgaattccagcacactggcggccgttactagtggatccgagctcggtac

SEQ ID NO: 5 - DNA, 857 bp, Artificial
  Synthetic primer
ttatgtcacaccacagaagtaaggttccttcacaaagatcccaagctgtcgatcgacatttctagaggatctcggacccggggaatccccgtcccccaacatgtccagatcgaaatcgtctagcgcgtcggcatgcgccatcgccacgtcctcgccgtctaagtggagctcgtcccccaggctgacatcggtcgggggggcggatctcggacccggggaatccccgtcccccaacatgtccagatcgaaatcgtctagcgcgtcggcatgcgccatcgccacgtcctcgccgtctaagtggagctcgtcccccaggctgacatcggtcgggggggcggatcccccgggctgcaggaattccggcgatacagtcaactgtctttgacctttgttactactctcttccgatgatgatgtcgcacttattctatgctgtctcaatgttagaggcatatcagtctccactgaagccaatctatctgtgacggcatctttattcacattatcttgtacaaataatcctgttaacaatgcttttatatcctgtaaagaatccattttcaaaatcatgtcaaggtcttctcgaggaaaaatcagtagaaatagctgttccagtctttctagccttgattccacttctgtcagatgtgccctagtcagcggagaccttttggttttgggagagtagcgacactcccagttgttcttcagacacttggcgcacttcggtttttctttggagcacttgagctttttaagtcggcaaatatcgcatgcttgttcgatagaagacagtagcttcatctttcaggaggctagggccgccagtgtgatggatatctgcagaattcgccctt

SEQ ID NO: 6 - DNA, 4726 bp, Homo sapiens
atgtccctgccacatgagtgcccggggccaggtacctgcaaggatgagaaacgcacacgtaaactgacgcccgtccgcccggaacacactgcactaccccgccaaaaagcacttccaagagtcctcggcgtgtcctccacacgaacacaatgctgtgcgaccggcaggagggtgactgggggtggttcatcacacggacgcagtgatctgcaaccgacaggacggtgactggggtgggggttcatcacacggacgcagtgctttgcgaccggcaggatggtgactggggggggttcatatcccacccccactcctcaggagcccagtatctgtcccccgcaccccccaggcctggtcacactctgcccacagcagagtgcaggggtggaggcagccggtacccagctgggtgaattgacgggccttgccatataccctcgggccacctttctgaactgggcagacagcttctctgggaacacgatgggagaacgctgctcccagccctaacctgccgtgccaccttggcaaatcgtgggacctttgcagctttggtttgcacccccagtgtaaaatatagacagtgccatcggctggcctagctcacaggtgtgcgggccggataaccgtgacaagcagagaccacaccacacagacaaaggctgtggccccagggcaggggcgtttgaggttagcctccgtcacttgctccatgggaatccagacggagttggggatgggctggaggctctcagtgctctgtggtctcccacacatgtcccctggaatcgagtcacggccctgtgaggtttttttgtttgtttctttttttgagacagagtcttgctctgtcgcccaggctggaatgcagtggtgcaatctcggctcactgcaacctctgcctcctgggttcaagctattctccagcctcagcctcccgagtagctgggactagaggcacgcgccaccacacccggctaatgtttgtatttttagtagagacggagtttccctgtttcccaggcttgtctacaactcttgacctcgtgatttgcccgccttggcctcccaaagtgctgggattacaggcgtgagccaccaagcccggccgctgccgctcactttctgtggaagccctcagggtgctggacgagtctccaacctccttgggagccagccagggctcccctgtagctggcccagccctcgacaggctgcagagagcagggccccgctggccgctgatcaccagcactggcagccagggccacctgggaggtttggagcatggagagccggagctgcgtgcccgcacgcctgaggctgacacagggcctcacacctgaatcagaagaacccccgcagtggggacagcagtgcggggccagccccggccctgtcttcccttgtccttccttgaccctcggtcccaccgtgcgtcaaccacagccccagaagggaaaagacgcggagtggccgcctgaccctcaggcctgctctcaggagaggaaggaagcgggacacctgcagaggggccgggggccccagtctccggttccccttcagcggcccgggcttgctgagccctcaaagtaggacacgcagccagagacacctgtggcctcacaaggcccgcttgccccccatggggtgcctggccccgcaagcgcgaagcggtcagaaaagggaggccgcgcttcagggctcaggccgcccaaggggccacgattcgaggaatcactcgggagggaggagcgcgcgagggccgggctgagcccccgcccgtgggtgcgcgcctggaaaactcgcagcctccgccctgggaagtggggcagcggctccctttgtccgcaccacaccaggtgggtgtcccggccgactccgctcgccacgtggccgcgcccctcccctccgccgagctccagcggcctggagtgcgctcgcccgttcggccctgcgcctcgtccgggtccccgggaagctgctaaggaggggccgtcgggtgggtggacgtccgtctcgggtctgggttccccgccgcacccgcgaggaaaaccggggatgcgctgcgatgaccggcagtaaccccggccggggcgcccgcggccggggtcaacgcccgcacttgcgccgcggcccgcgaggcatccgggaccgccccgccgcccgcccatcaccctatcgcctagcaacgcccacccgcgcgccgccattgggccggtgcgaacacccctggcgcccgattggttgctgctgccgccgcgccgcgcaacgccgcgccctcagccagctccgcgcgggcagcgacggcggcgtggagtcggcggccgaagtgcgggcacgcgggtccgtccgtccttccgcgcgcctgtgggtccgagcgccccgccgcagccggctggacccccggtgtggccacggggtagtccccagtgtggcccaagcattcccatcccgcacacgtggccccggcggacacgggggtcggggatggctcgggaggcgcgccgcgggctcggtcgcagccgcgtgcgcgtcggggcccgcggggcgggaggcggaagtcggggggcggtggtttcccgcccgccccgcccccggcgctcctcagtcccagccccacccccccaggcgcgttcccgcgcagggtcccggctcgggcggcggcgcgcacgagcatcaccccaccggcggcggcgcgccgggtcccggggcgcagccccgacgctcattggcctgggcggcgcagccaagctgtcagcccatgtggcgtggccgcccggggatggccgcggtttaaatagcgtcggcgccggcctagagggagccagagagacggcagcggccccggcctccctctccgccgcgcttcagcctcccgctccgccgcgctccagcctcgctctccgccgcccgcaccgccgcccgcgccctcaccaggtacgagcagccggcgctggggccggggcgcgggtcgtggtcggggctgggcccggggttcccggggtggcccggctcctcatcctccgctctcggggcgcgcgtggcccggccccgtatccaggggtcgcctgggcggcctgtccgggtctcggtgttcgtgccccttcggaggcgcccggggccccgcggagtaggtgggcggatggcgggggaaggacgccggggaaagccaccaacagccgccttcgcttcccgtgggggaggccgcggaccctgaagggcctggaggagcccccggctcggcctccccgcgcccacaaaggcgccgccgcatcctcctcccggagccgccccggagccgaggcccagcccgaaggggacgccgcgagggaggaacgggggggcgcaggggccgcgccgggaccggcctccccatcacgtggcgcagcccggccgaagcgcaggggtcccgagcagcccccgcccggggcgaagggaggccgggcgcgccgcggtgttcccaccccctgcctggccgtcgaccccgcgtgaatagcaagcggcgaggacccagcgtgcgggcctggagctggcccggggcagccaagcctctgtgaatcgcagcgggtgggaaggcggccgcctccctgccgggacggcccaagggaggccgcactgccgaggggcggcgcgcgtatcaggccctggggcctcgtgcaagtggccagaccagcgctggcctccgccccctccccccagggccgcaccagccctcatgctggaggaggagccggcggcctggcacccgttccgggtcttagggacctgggctccagggagggcctcctgtggtgtgtgggttggtatggggaggcaagcgtctgtgggttcacccgcgcaggtgaagggtgccaggcaacacctgcgtgtcctggccaggagtggttctccaggttggaccctgctggaggggttggactggctgggtcggtccacagtggggcaggggccacctttcccccagagcataggctatccccatttcgtccctccccagccctggtggctcttgggctggcctggacagcgggcccccaccccaacacgggtgagcatgggtgctgagtacccccctctagggctcccagccacctccatgccccggctttgggaccctgcctctccatttctttgatggcccttagcaggctggccacctgaggcttgtccccatctcaggtgaccctgcctgctcctcactgtgagccccctggtgccctagggatggcctgggacaagctccaggagccttggaaggctgagactcaggcgagtggaagccgacacaggcgcagaaaccagtaactgctggggaggccagggctgcagggctccagtggggtaccccagccaataccctgaactgaatgggtcagggcccagaggcgggcctgttcccagagcacgcagcccctcccagcgagagcggggtcagggcccacgaactggccacatgaggaagggagccgcaggagccagggcccgcacggaccctcagggtggtgcttggccccatgggtgtccacctgttctgggtgccgaggctgttgacgggggtgtcgtggccgggatgtgtggggcactcacaccgcccgccctgcagagcagccatggaggaggtggtgattgccggcatgtccgggaagctgccagagtcgagaacttgcaggagttctgggacaacctcatcggcggtgtggacatggtcacggacgatgaccgt

SEQ ID NO: 7 - DNA, 1846 bp, Homo sapiens
aaaagtgcctttgttggcctgggctcaggaatccagagaaactggtcaggaggaggccccagtgacaaaaacccctccctctgcccccgcccctctgccagagccatataactgctcaacctgtccccgagagagagtgccctggcagctgtcggctggaaggaactggtctgctcacacttgctggcttgcgcatcaggactggctttatctcctgactcacggtgcaaaggtgcactctgcgaacgttaagtccgtccccagcgcttggaatcctacggcccccacagccggatcccctcagccttccaggtcctcaactcccgtggacgctgaacaatggcctccatggggctacaggtaatgggcatcgcgctggccgtcctgggctggctggccgtcatgctgtgctgcgcgctgcccatgtggcgcgtgacggccttcatcggcagcaacattgtcacctcgcagaccatctgggagggcctatggatgaactgcgtggtgcagagcaccggccagatgcagtgcaaggtgtacgactcgctgctggcactgccgcaggacctgcaggcggcccgcgccctcgtcatcatcagcatcatcgtggctgctctgggcgtgctgctgtccgtggtggggggcaagtgtaccaactgcctggaggatgaaagcgccaaggccaagaccatgatcgtggcgggcgtggtgttcctgttggccggccttatggtgatagtgccggtgtcctggacggcccacaacatcatccaagacttctacaatccgctggtggcctccgggcagaagcgggagatgggtgcctcgctctacgtcggctgggccgcctccggcctgctgctccttggcggggggctgctttgctgcaactgtccaccccgcacagacaagccttactccgccaagtattctgctgcccgctctgctgctgccagcaactacgtgtaaggtgccacggctccactctgttcctctctgctttgttcttccctggactgagctcagcgcaggctgtgaccccaggagggccctgccacgggccactggctgctggggactggggactgggcagagactgagccaggcaggaaggcagcagccttcagcctctctggcccactcggacaacttcccaaggccgcctcctgctagcaagaacagagtccaccctcctctggatattggggagggacggaagtgacagggtgtggtggtggagtggggagctggcttctgctggccaggatagcttaaccctgactttgggatctgcctgcatcggcgttggccactgtccccatttacattttccccactctgtctgcctgcatctcctctgttccgggtaggccttgatatcacctctgggactgtgccttgctcaccgaaacccgcgcccaggagtatggctgaggccttgcccacccacctgcctgggaagtgcagagtggatggacgggtttagaggggaggggcgaaggtgctgtaaacaggtttgggcagtggtgggggagggggccagagaggcggctcaggttgcccagctctgtggcctcaggactctctgcctcacccgcttcagcccagggcccctggagactgatcccctctgagtcctctgccccttccaaggacactaatgagcctgggagggtggcagggaggaggggacagcttcacccttggaagtcctggggtttttcctcttccttctttgtggtttctgttttgtaatttaagaagagctattcatcactgtaattattattattttctacaataaatgggacctgtgcacaggaaaaaaaaaaaaaaaaaaaaaaaa

(19) United States
(12) Patent Application Publication
(10) Pub. No.: US 2009/0192101 A1
(43) Pub. Date: Jul. 30, 2009

(54) CANCER-SPECIFIC PROMOTERS

(76) Inventors: Mien-Chie Hung, Houston, TX (US); Xiaoming Xie, Houston, TX (US); Jing-Yu Lang, Houston, TX (US)

Correspondence Address: FULBRIGHT & JAWORSKI, LLP, 1301 MCKINNEY, SUITE 5100, HOUSTON, TX, 77010-3095, US

(21) Appl. No.: 11/943,057

(22) Filed: Nov. 20, 2007

Related U.S. Application Data
(60) Provisional application No. 60/860,745, filed on Nov. 22, 2006.

(51) Int. Cl. A61K 31/7088; C07H 21/04; A61P 35/00

(52) U.S. Cl. 514/44; 536/23.5

(57) ABSTRACT

The present invention regards cancer-specific control sequences that direct expression of a polynucleotide encoding a therapeutic gene product for treatment of the cancer. Specifically, the invention encompasses breast cancer-specific and ovarian cancer-specific control sequences. Two breast cancer-specific sequences utilize specific regions of fatty acid synthase and claudin 4 promoters, particularly in combination with a two-step transcription amplification sequence and/or a post-transcriptional control sequence. Two ovarian cancer-specific sequences utilize specific regions of hTERT and survivin promoters, particularly in combination with a two-step transcription amplification sequence and/or a post-transcriptional control sequence. In more particular embodiments, these polynucleotides are administered in combination with liposomes.

The present invention claims priority to U.S. Provisional Patent Application No. 60/860,745, filed Nov. 22, 2006, which is incorporated by reference herein in its entirety.

### FIELD OF THE INVENTION

The present invention is directed at least to the fields of cell biology, molecular biology, cancer biology, and medicine. More particularly, the present invention regards cancer-specific regulatory sequences for regulation of expression of a therapeutic polynucleotide useful for cancer therapy.

### BACKGROUND OF THE INVENTION

The ability to control expression of particular polynucleotides upon gene transfer is a useful function, particularly for applications where specific localized activity is desired. Such is the case for cancer, where it is prudent to confine destructive or lethal gene products to the cancerous cells while preventing at least in part such activity in normal cells.

Current cancer therapies, such as chemotherapy (CT) and radiotherapy, have low selectivity for tumor cells and side effects for normal tissues. To minimize the side effects, these therapies are generally given in an intermittent manner, allowing normal cells to recover between treatment cycles. However, during the recovery period, some surviving cancer cells become more resistant to the treatment because of gene mutation. Consequently, cancer recurrence or progression may occur. Tumor-targeting gene therapy minimizes treatment side effects and the risk of developing resistance by acting on the tumor-specific signaling pathways. The present invention provides a long-felt need in the art to provide breast and ovarian tissue-specific expression of gene sequences to facilitated targeted gene therapy.

### SUMMARY OF THE INVENTION

The present invention provides novel tissue-specific promoters for regulation of expression of a therapeutic polynucleotide. These therapeutic compositions and methods that utilize them are helpful for cancer treatment, and a skilled artisan recognizes that any additional means in an arsenal to fight cancer is beneficial to public health.

In particular, the invention provides compositions, such as therapeutics, and methods of using compositions directed to cancer-specific regulated expression of a therapeutic polynucleotide in gene therapy for cancer, such as at least ovarian and breast cancer, for example.

Thus, the present invention generally relates to methods for inhibiting proliferation in a cancer cell and/or tumor cell, the method comprising contacting the cell with a therapeutic polypeptide in an amount effective to inhibit proliferation utilizing a cancer-specific promoter, such as one described herein. Inhibition of proliferation may be indicated by, for example, an induction of apoptosis of a cell, such as, for example, in cell culture, inhibition of growth of a cancer cell line, reduction in size of a tumor, and/or an increase in survivability, in exemplary embodiments. More preferably, in some embodiments the cell in which proliferation is to be inhibited is a cell in a living organism, for example a human. The inhibition of such transformation has great utility in the prevention and/or treatment of such transformation-driven events as cancer, tumorigenesis, and/or metastasis.

The present invention encompasses polynucleotide constructs comprising control sequences that direct expression of a therapeutic polynucleotide in a particular tissue and/or type of cell. The polynucleotide may be contacted with or introduced to a cell through any of a variety of manners known to those of skill. The therapeutic polynucleotide may be introduced through direct introduction of the polynucleotide to a cell or tissue of interest. In this case, the therapeutic polynucleotide may be obtained through any method known in the art.

In specific aspects of the invention, RNA or DNA comprising the therapeutic polynucleotide may be introduced to the cell by any manner known in the art. In certain preferred embodiments, the therapeutic polynucleotide is introduced into the cell through the introduction of a DNA segment that encodes the therapeutic gene product. In some such embodiments, it is envisioned that the DNA segment comprising the therapeutic polynucleotide is operatively linked to the inventive control sequences. The construction of such gene/control sequence DNA constructs is well-known within the art and is described in detail herein.

In certain embodiments for introduction, the DNA segment may be located on a vector, for example, a plasmid vector or a viral vector. The virus vector may be, for example, retrovirus, adenovirus, herpesvirus, vaccina virus, and adeno-associated virus. Such a DNA segment may be used in a variety of methods related to the invention. The vector may be used to deliver a mutant bik polynucleotide to a cell in one of the gene-therapy embodiments of the invention, in specific embodiments. Also, such vectors can be used to transform cultured cells, and such cultured cells could be used, inter alia, for the expression of mutant Bik in vitro, for example.

A skilled artisan recognizes that the promoters of the invention are useful in any context, including non-cancerous cell-specific expression or even expression of a polynucleotide that is not cell- or tissue-specific in nature.

In a particular embodiment, a therapeutic gene product is effective on the respective breast or ovarian cancer tissue. In exemplary embodiments, the present invention is useful for delivering genetic constructs that treat cancers that are estrogen receptor positive, EGF receptor overexpressing, Her2/neu-overexpressing, Her-2/neu-nonoverexpressing, Akt overexpressing, androgen dependent, and/or angrogen independent, for example. That is, the therapeutic gene product is effective on the respective cancer cells regardless of their status of oncogene overexpression, such as Her-2/neu, EGFR, AKT, or regardless of whether their growth is hormone dependent or not.

A skilled artisan is aware of publicly available databases that provide promoter or therapeutic polynucleotide sequences, such as the National Center for Biotechnology Information's GenBank® database or commercially available databases, such as from Celera Genomics, Inc. (Rockville, Md.). Although there are a plethora of therapeutic polynucleotides that are known in the art that are later discovered that may be utilized in the invention, some examples include inhibitors of cellular proliferation, regulators of programmed cell death, tumor suppressors and antisense sequences of inducers of cellular proliferation. The therapeutic polynucleotide may encode small interfering RNAs or antisense sequences, for example. Particular exemplary therapeutic polynucleotides include those that encode mutant Bik, retinoblastoma, Blk, IL-12, IL-10, IFN-α, cytosine deaminase, GM-CSF, E1A, p53, and other pro-apoptotic proteins, for example. Also, a construct may comprise such therapeutic polynucleotides as TNFα or p53 or inducers of apoptosis including, but not limited to, Bik, p53, Bax, Bak, Bcl-x, Bad, Bim, Bok, Bid, Harakiri, Ad E1B, Bad and ICE-CED3 proteases. In specific aspects of the invention, a mutant Bik polynucleotide encoding an amino acid substitution at threonine 33, serine 35, or both, in reference to wildtype Bik, is utilized. In particular aspects of these embodiments, the amino acids of the mutant Bik polypeptide are substituted with aspartate. In other particular aspects, one or more phosphorylation sites are defective in a mutant Bik. In additional embodiments, the mutant Bik retains anti-cell proliferative and/or pro-apoptotic activity. In specific aspects, the therapeutic polynucleotide is E1A.

In particular embodiments, a construct comprising the inventive therapeutic polynucleotide and respective cancer-specific control sequences is introduced into a cell that is a human cell. In many embodiments, the cell is a tumor cell. In some presently preferred embodiments, the tumor cell is a breast tumor cell, an ovarian tumor cell, a prostrate tumor cell, or a pancreatic tumor cell. In some embodiments, a construct comprising the therapeutic polynucleotide and respective cancer-specific control sequences is introduced by injection. In particular embodiments, the construct comprising the therapeutic polynucleotide and respective cancer-specific control sequences is comprised in a liposome.

In some embodiments of the present invention, a construct comprising the therapeutic polynucleotide and respective cancer-specific control sequences is used in combination with other anti-transformation/anti-cancer therapies. These other therapies may be known at the time of this application, or may become apparent after the date of this application. A construct comprising the therapeutic polynucleotide and respective cancer-specific control sequences may be used in combination with other therapeutic polypeptides, polynucleotides encoding other therapeutic polypeptides, chemotherapeutic agents, surgical methods, and/or radiation, for example.

A construct comprising the therapeutic polynucleotide and respective cancer-specific control sequences may be used in conjunction with any suitable chemotherapeutic agent. In one representative embodiment, the chemotherapeutic agent is Taxol, for example. A construct comprising the therapeutic polynucleotide and respective cancer-specific control sequences also may be used in conjunction with radiotherapy. The type of ionizing radiation constituting the radiotherapy may comprise x-rays, γ-rays, and microwaves, for example. In certain embodiments, the ionizing radiation may be delivered by external beam irradiation or by administration of a radionuclide. The cancer-specific control sequence-regulated therapeutic gene product also may be used with other gene-therapy regimes. In particular embodiments, the construct comprising the therapeutic polynucleotide and respective cancer-specific control sequences is introduced into a tumor. The tumor may be in an animal, in particular, a mammal, such as a human.

Constructs having the inventive tissue-specific promoters regulating expression of a therapeutic gene product and polynucleotides of the present invention may also be introduced using any suitable method. A “suitable method” of introduction is one that places a therapeutic gene product under conditions, such as in a position, to reduce the proliferation of a tumor cell, preferably in the tissue or cells of interest and/or to ameliorate at least one cancer symptom. For example, injection, oral, and inhalation methods may be employed, with the skilled artisan being able to determine an appropriate method of introduction for a given circumstance, and the tissue-specific control sequences of the present invention direct expression of the therapeutic polynucleotide at least primarily in the tissue or cells of interest. In the embodiments where injection will be used, this injection may be intravenous, intraperitoneal, intramuscular, subcutaneous, intratumoral, and/or intrapleural, for example, or of any other appropriate form.

In certain other aspects of the present invention, there are provided therapeutic kits comprising in a suitable container a pharmaceutical formulation of a construct comprising the inventive control sequences. In additional aspects, a polynucleotide comprising the inventive control sequences comprises one or more cloning sites such that a desired polynucleotide, such as a polynucleotide of interest, may be cloned into the site. In particular embodiments, in a polynucleotide having a 5′ to 3′ orientation the one or more cloning sites may be located 5′ of control sequence or 3′ of the control sequence. In additional aspects, one or more therapeutic polynucleotides are also comprised in the kit, such as on the same nucleic acid molecule as the control sequences of the present invention. Such a kit may further comprise a pharmaceutical formulation of a therapeutic polypeptide, polynucleotide encoding a therapeutic polypeptide, and/or chemotherapeutic agent. One or more primers to amplify a regulatory sequence and/or a therapeutic polynucleotide may be provided in the kit.

The anti-tumor activity, anti-cell proliferation activity, and/or pro-apoptotic activity provided by the gene product of the therapeutic polynucleotide may be useful for an organism other than the one from which the therapeutic polynucleotide is derived. For example, a murine therapeutic polynucleotide may be used alternatively or in addition for human treatment.

Thus, the present invention provides cancer-specific control sequences for targeted expression of a therapeutic polynucleotide, and, therefore, the present invention is directed to a novel improvement to the overall arts of cell growth control, including inhibition of cell proliferation and/or facilitation of cell death. In a specific embodiment, the inhibition of a cell proliferation comprises a delay in its rate of proliferation, a delay in its total cell numbers of proliferation, or both.

In an additional object of the present invention, there is a method of treating and/or preventing growth of a cell in an individual comprising the step of administering to the individual a construct comprising cancer-specific control sequences that regulate expression of a therapeutic polynucleotide. In another specific embodiment, the administration of the construct comprising the inventive controls sequences is by a liposome.

In another object of the present invention, there is a method of treating and/or preventing growth of a cell in an individual comprising the step of administering to the individual a nucleic acid comprising a tissue-specific control sequence encompassed by the present invention. In another specific embodiment, the administration of the nucleic acid is by a vector selected from the group consisting of a plasmid, a retroviral vector, an adenoviral vector, an adeno-associated viral vector, a liposome, and a combination thereof. The composition comprising the nucleic acid may be dispersed in a pharmacologically acceptable excipient, and the composition may be administered to an animal having a proliferative cell disorder.

In other particular embodiments, the control sequence is operably linked to a polynucleotide encoding a therapeutic gene product, such as one that is an inhibitor of cell proliferation, a regulator of programmed cell death, or a tumor suppressor, or one encompassing two of more of these activities. Constructs of the present invention may be comprised in a liposome.

In a further object of the invention, a therapeutic polynucleotide is regulated by a tissue-specific promoter, such as one that targets cancerous tissue. Although any promoter that targets cancerous tissue preferentially over non-cancerous tissue, in a specific embodiment the cancer-specific promoter is a breast cancer specific promoter or an ovarian-specific promoter, for example, or it may be useful for both breast and ovarian-specific expression, in specific embodiments.

In a particular embodiment, a breast cancer-specific promoter comprises a breast cancer-specific sequence and additional specific regulatory elements. In a particular embodiment, the tissue-specific sequence comprises fatty acid synthase or claudin 4 regulatory sequence. The inventors show herein that the inventive composite promoters drive gene expression selectively in breast cancer cells. They are useful for gene targeting to target and treat primary and metastatic breast cancers with less toxicity to normal tissues. In specific embodiments, the additional specific regulatory elements comprise a post-transcriptional regulatory element and/or a two-step transcriptional amplification (TSTA) sequence.

In another specific embodiment of the present invention, a breast cancer-specific promoter regulates expression of a therapeutic polynucleotide in which the promoter comprises the post transcriptional regulatory element of the woodchuck hepatitis virus (WPRE) and/or a TSTA element, for example. This promoter can be used to specifically drive gene expression of a therapeutic polynucleotide in breast cancer in vivo or in vitro.

In particular embodiments, constructs of the present invention comprise an enhancer, such as cytomegalovirus (CMV) enhancer, Glyceraldehyde-3-phosphate dehydrogenase promoter (GAPDH), or the β-actin promoter. The construct may further comprise a post-transcriptional regulatory sequence, such as, for example, woodchuck hepatitis virus post-transcriptional regulatory element (WPRE). In additional embodiments, a construct of the present invention comprises a TSTA sequence, wherein the TSTA sequence includes a DNA binding domain, such as Gal1, Gal4, or LexA, for example, and an activation domain, such as VP2 or VP16, for example. In particular aspects of the invention, the TSTA sequence is GAL4-VP2 or GAL4-VP16, for example. The DNA-binding domain and activation domain are operably linked.

In another object of the invention, a polynucleotide construct comprises a breast cancer-specific control sequence that comprises at least two of the following sequences: a breast tissue-specific control sequence; a cancer-specific control sequence; a post-transcriptional regulatory sequence; and a two-step transcriptional amplification (TSTA) sequence, said TSTA sequence including a DNA binding domain and an activation domain.

In a specific aspect of the invention, a polynucleotide construct that comprises an ovarian cancer-specific control sequence further comprises a post-transcriptional regulatory sequence, such as woodchuck hepatitis virus post-transcriptional regulatory element (WPRE) sequence. The control sequence is operably linked to a polynucleotide encoding a therapeutic gene product, in some embodiments, such as an inhibitor of cell proliferation, a regulator of programmed cell death, or a tumor suppressor, or one encompassing two or more of these activities. The polynucleotide construct comprising a breast or an ovarian cancer-specific control sequence may be comprised in a liposome.

In an additional object of the invention, there is a polynucleotide construct comprising an ovarian or breast cancer-specific control sequence comprising: a respective ovarian or breast tissue-specific control sequence and a two-step transcriptional amplification (TSTA) sequence, said TSTA sequence including a DNA binding domain and an activation domain. In the polynucleotide construct comprising a breast cancer-specific or ovarian cancer-specific control sequence, the DNA binding domain of the TSTA can be Gal1, Gal4, or LexA, and the activation domain of the TSTA can be VP2 or VP16. In particular, the TSTA sequence is GAL4-VP2 or GAL4-VP16.

In an additional object of the invention, there is a method of inhibiting breast cancer cell proliferation, comprising contacting a breast cancer cell with an effective amount of a polynucleotide construct that comprises a selected portion of the breast tissue-specific promoter, wherein the selected portion may be operably linked to a polynucleotide encoding a gene product effective to inhibit the cell proliferation. In particular aspects of the invention, the construct further comprises an enhancer, such as CMV, Glyceraldehyde-3-phosphate dehydrogenase promoter (GAPDH), or the β-actin promoter.

In another object of the invention, there is a method of inhibiting breast cancer cell proliferation, comprising contacting a breast cancer cell with an effective amount of a polynucleotide construct having at least two of the following sequences: a breast cell-specific control sequence; a two-step transcriptional amplification sequence; and a cancer cell-specific sequence, wherein the sequences are operably linked to a polynucleotide encoding a gene product effective to inhibit the breast cancer cell proliferation. The construct may further comprise a post-transcriptional control sequence operably linked to the polynucleotide encoding a gene product effective to inhibit the breast cancer cell proliferation, such as a WPRE sequence, for example.

In an additional object of the invention, there is a method of inhibiting breast cancer cell proliferation, comprising contacting a breast cancer cell with an effective amount of a polynucleotide construct comprising a breast cell-specific sequence and a two-step amplification sequence, both of which are operably linked to a polynucleotide encoding a gene product effective to inhibit the cell proliferation. The construct may further comprise a post-transcriptional control sequence operably linked to the polynucleotide encoding a gene product effective to inhibit the cell proliferation, such as a WPRE sequence, for example.

In a further object of the invention, there is a method of treating breast cancer in an individual having the cancer, comprising contacting at least one breast cancer cell of the individual with a therapeutically effective amount of a polynucleotide construct comprising a portion or all of a breast tissue-specific promoter, wherein the selected portion is operably linked to a polynucleotide encoding a gene product effective to treat breast cancer. The construct may further comprise an enhancer, such as CMV enhancer, and the polynucleotide may be comprised in a liposome.

In an additional object of the invention, there is a method of inhibiting ovarian cancer cell proliferation, comprising contacting an ovarian cancer cell with an effective amount of a polynucleotide construct that comprises a selected portion of the ovarian tissue-specific promoter, wherein the selected portion may be operably linked to a polynucleotide encoding a gene product effective to inhibit the cell proliferation. In particular aspects of the invention, the construct further comprises an enhancer, such as CMV, Glyceraldehyde-3-phosphate dehydrogenase promoter (GAPDH), or the β-actin promoter, for example.

In another object of the invention, there is a method of inhibiting ovarian cancer cell proliferation, comprising contacting a ovarian cancer cell with an effective amount of a polynucleotide construct having at least two of the following sequences: an ovarian cell-specific control sequence; a two-step transcriptional amplification sequence; and a cancer cell-specific sequence, wherein the sequences are operably linked to a polynucleotide encoding a gene product effective to inhibit the ovarian cancer cell proliferation. The construct may further comprise a post-transcriptional control sequence operably linked to the polynucleotide encoding a gene product effective to inhibit the ovarian cancer cell proliferation, such as a WPRE sequence, for example.

In an additional object of the invention, there is a method of inhibiting ovarian cancer cell proliferation, comprising contacting an ovarian cancer cell with an effective amount of a polynucleotide construct comprising an ovarian cell-specific sequence and a two-step amplification sequence, both of which are operably linked to a polynucleotide encoding a gene product effective to inhibit the cell proliferation. The construct may further comprise a post-transcriptional control sequence operably linked to the polynucleotide encoding a gene product effective to inhibit the cell proliferation, such as a WPRE sequence, for example.

In a further object of the invention, there is a method of treating ovarian cancer in an individual having the cancer, comprising contacting at least one ovarian cancer cell of the individual with a therapeutically effective amount of a polynucleotide construct comprising a portion or all of an ovarian tissue-specific promoter, wherein the selected portion is operably linked to a polynucleotide encoding a gene product effective to treat ovarian cancer. The construct may further comprise an enhancer, such as CMV enhancer, and the polynucleotide may be comprised in a liposome.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings.

FIG. 1 shows transient luciferase expression of fatty acid synthase promoter in human normal and cancer cell lines.

FIG. 2 shows transient luciferase expression of claudin 4 promoter in human normal and cancer cell lines.

FIG. 3 shows the promoter activities of VISA (VISA=WPRE element+TSTA sequence)-enhanced claudin 4 and fatty acid synthase in breast cancer cells and normal cells in vitro.

FIGS. 4A and 4B show activity of selective promoters in ovarian cancer cell lines and normal cells.

FIGS. 5A and 5B show comparison of CMV, hTert-VISA (TV), and survivin-VISA (SUV) promoter activities in ovarian cancer cell lines and normal cells.

FIG. 6 shows hTert-VISA (TV) is specifically expressed in ovarian cancer cells but not in normal cells.

FIG. 7 shows the activities of fatty acid synthase (FASN) promoter in human normal and cancer cells. 1×10⁶ cells were transfected with 2 μg pGl3-FASN-luciferase vector, as well as 0.2 μg pRL-TK as internal standards by electroporation. The luciferase activity was measured after 24 hrs.

FIG. 8 shows the promoter activities of tight junction protein Claudin 4 in human normal and cancer cells. 1×10⁶ cells were transfected with 2 μg pGl3-claudin 4 luciferase vector, as well as 0.2 μg pRL-TK as internal standards by electroporation. The luciferase activity was measured after 24 hrs.

FIG. 9 demonstrates the promoter activities of Claudin 4 and fatty acid synthase in human normal and cancer cells. 1×10⁶ cells were transfected with 2 μg pGl3-claudin 4 luciferase vector, as well as 0.2 μg pRL-TK as internal standards by electroporation. The luciferase activity was measured after 24 hrs.

FIG. 10 shows the activities of VISA-enhanced claudin 4 and fatty acid synthase promoters in breast cancer and other cell lines. 1×10⁶ cells were transfected with 2 μg pGL3-VISA-Claudin4-Luc or pGL3-VISA-FASN-Luc plasmid, as well as 0.2 μg pRL-TK as internal standards by electroporation. The luciferase activities were measured 24 hrs after transient transfection.

FIG. 11 provides that 0.5-1×10⁴ cells were transfected with indicated concentration plasmid by electroporation assay. The cells were incubated with thiazolyl blue tetrazolium bromide for 4 hrs after 72 hrs, and dissolved with DMSO for 10 min, and measured at OD₅₇₀ nm.

FIG. 12 shows the activities of VISA-enhanced claudin 4 and fatty acid synthase promoters in human breast cancer cell lines. 1×10⁶ cells were transfected with 2 μg VISA-claudin 4 promoter vector, as well as 0.2 μg pRL-TK as internal standards by SN liposome transfection. The luciferase activity was measured at indicated times.

FIG. 13 relates to activity of VISA-Claudin4-Luc. In FIG. 13A, the activities of VISA-enhanced claudin 4 were selectively expressed in 4T1 breast cancer, while CMV promoter was strongly expressed in lung in vivo. In FIG. 13B, the VISA-Claudin4-Luc was strongly expressed in breast carcinoma, while expressed very weakly in other organs of mice. In FIG. 13C, the luciferase expression of VISA-Claudin4-Luc and CMV-luc in lung and tumor were measured by IVIS 100 imaging system, and the data were averaged by 5 mice in each group. 50 μg plasmid plus HLDC liposome were administered into mice by tail vein for one time, and mice were underwent imaging for 1 min with the noninvasive imaging system (IVIS imaging system, xenogen, Alameda, Calif.) after 48 hrs treated with D-luciferins.

FIG. 14 shows the acute toxicity of pUK21-VISA-Claudin4-BIKDD (FIG. 14A) and pUK21-VISA-FASN-BIKDD (FIG. 14B) in normal BALB/cA mice. Each mice was injected with indicated concentration plasmid plus HLDC liposome by tail vein, and mice survival were recorded in 14 days.

FIG. 15 concerns VISA-Claudin4-BIKDD treatment. In FIG. 15A, the tumor growth of MDA-MB-435 orthotopic xenografts was significantly suppressed by VISA-Claudin4-BIKDD treatment. In FIG. 15B, the tumor growth of 4T1 orthotopic synergic tumor model was significantly suppressed by VISA-Claudin4-BIKDD treatment. 2×10⁶ cells were incubated to the mammary fat pad of female athymic mice or BALB/Ac mice, and the mice were treated with indicated concentration of HLDC and plasmid mixture when the tumor volume reached to 50 mm³. Plasmid plus HLDC liposome were administered into mice by tail vein, and mice were measured tumor volume twice per week, and calculated as following: tumor volume=0.5×length×width×width.

FIG. 16 demonstrates that VISA-Claudin4-BIKDD greatly prolonged the survival time of MDA-MB-435-Luc orthotopic xenografts in vivo.

FIG. 17 shows that VISA-Claudin4-BIKDD has additive combination efficacy with lapatinib and taxol in MDA-MB-453 breast cancer cell line.

FIG. 18 provides that VISA-Claudin4-BIKDD has additive combination efficacy with lapatinib and taxol in MDA-MB-468 breast cancer cell line.

FIG. 19 shows that VISA-Claudin4-BIKDD has additive combination efficacy with lapatinib and taxol in BT474 breast cancer cell line.

FIG. 20 demonstrates that VISA-Claudin4-BIKDD does not promote the cytotoxicity of lapatinib and taxol in MCF10A human breast normal cell line.

FIG. 21 demonstrates that hTERT and Survivin promoters are active in ovarian cancer. In FIG. 21A, there is a diagram of the promoter-driven luciferase report plasmids. In FIG. 21B, there is a panel of ovarian cancer cell lines, normal ovarian epithelia cells (NOE115) and fibroblasts (WI-38) that were transiently cotransfected with plasmid DNA indicated and pRL-TK. 48 h later, dual luciferase ratio was measured and shown as RLU (ratio) normalized to the Renilla luciferase control. The data represent the mean of four independent experiments. Bar, SD.

FIG. 22 demonstrates that T-VISA is robust in ovarian cancer cell lines. FIG. 22A: Schematic diagram of engineered hTERT-VISA constructs in the pGL3 backbone. FIG. 22B: Ovarian cancer cell lines, normal ovarian epithelia cells (NOE115) and fibroblasts (WI-38) were transiently cotransfected with the indicated plasmid DNA and pRL-TK. Forty-eight hours later, dual luciferase ratio was measured and shown as RLU (ratio) normalized to the Renilla luciferase control. The data represent the mean of four independent experiments. Bar, SD.

FIG. 23 shows that T-VISA transcriptionally targets transgene expression to ovarian cancer cells in vivo. Female nude mice bearing orthotopic HeyA8 tumors were given 50 μg of DNA in a DNA:liposome complex via the tail vein. Two days later, mice were anesthetized and subjected to in vivo imaging for 2 min at 10 min after intraperitoneal injection of d-luciferin (FIG. 23A). HeyA8 tumors of mice from A were subjected to ex vivo imaging (FIG. 23B). The photon signals were quantified by Xenogen's Living Imaging software (shown on the right). Bars, SD; n=3 per group. CMV-Luc, pGL3-CMV-Luc; T-VISA-Luc, pGL3-hTERT-VISA-Luc; Ctrl, pGL3-C-VISA.

FIG. 24 shows cell killing activities of CMV-E1A, T-VISA-E1A in ovarian cancer cell lines and normal cells. A panel of ovarian cancer cell lines and normal fibroblasts were cotransfected with pUK21-T-VISA-E1A, pUK21-CMV-EIA, and negative control (pUK21-TV), plus 100 ng of pGL3-CMV-Luc. The signal was imaged with the IVIS system two days after transfection. The percentage of the signals as compared with the negative control (setting at 100%) was presented. The data represent the mean of three independent experiments. Bars, SD.

### DETAILED DESCRIPTION OF THE INVENTION

U.S. patent application Ser. No. 11/096,622 (U.S. Patent Publication US2005/0260643) is related in subject matter and is incorporated by reference herein in its entirety.

As used herein the specification, “a” or “an” may mean one or more. As used herein in the claim(s), when used in conjunction with the word “comprising”, the words “a” or “an” may mean one or more than one. As used herein “another” may mean at least a second or more. In specific embodiments, aspects of the invention may “consist essentially of” or “consist of” one or more sequences of the invention, for example. Some embodiments of the invention may consist of or consist essentially of one or more elements, method steps, and/or methods of the invention. It is contemplated that any method or composition described herein can be implemented with respect to any other method or composition described herein.

In some embodiments a polynucleotide comprising the inventive control sequences is delivered by, for example, either a viral or non-viral delivery system into an appropriate recipient animal to suppress tumor growth and development. In one exemplary embodiment of the present invention, the delivered therapeutic gene product acts through an apoptosis mechanism to suppress tumor growth and development.

In one aspect of the invention, a therapeutic polypeptide comprised in a construct including a tissue-specific control sequence is administered as a polynucleotide targeted for expression in breast cancer or ovarian cancer, for example. In certain aspects of the invention, a breast cancer-specific promoter or ovarian cancer-specific promoter controls expression of the therapeutic polynucleotide. As used herein, the term “therapeutic polynucleotide” refers to a polynucleotide that encodes a therapeutic gene product, which may be an RNA, protein, polypeptide, or peptide, for example.

In a specific embodiment, the control sequences of the present invention comprise a composite (chimeric) promoter. For example, breast cancer specific promoters comprised of a breast cancer specific regulatory sequence, such as, for example, an optional CMV promoter enhancer sequence linked with breast cancer specific segments in a variety of genes, may be utilized. Exemplary breast cancer specific promoters include the fatty acid synthase promoter and the promoter of tight junction protein claudin 4, which may be referred to as CLDN4. The inventive promoters drive gene expression selectively in breast cancer cells and possess activity levels comparable to the CMV promoter, in specific embodiments. Constructs employing the fatty acid synthase and/or claudin 4 chimeric promoters are used in gene transfer to target and treat primary and metastatic breast cancers with less toxicity to normal tissues, preferably by selectively killing breast cancer cells and/or significantly reducing breast tumor growth and/or growth rate.

In a specific embodiment, the control sequences of the present invention comprise a composite (chimeric) promoter. For example, ovarian cancer specific promoters comprised of an ovarian cancer specific regulatory sequence, such as, for example, CMV promoter enhancer sequence linked with ovarian cancer specific segments in a variety of genes, may be utilized. Exemplary ovarian cancer specific promoters include the hTERT promoter and the promoter of survivin. The inventive promoters drive gene expression selectively in breast cancer cells and possess activity levels comparable to the CMV promoter, in specific embodiments. Constructs employing the fatty acid synthase and/or claudin 4 chimeric promoters are used in gene transfer to target and treat primary and metastatic breast cancers with less toxicity to normal tissues, preferably by selectively killing breast cancer cells and/or significantly reducing breast tumor growth and/or growth rate.

In other aspects of the invention, a breast cancer-specific or ovarian cancer-specific promoter controls expression of a therapeutic polynucleotide. In a particular embodiment of the invention there is a composite breast cancer-specific or ovarian cancer-specific regulatory construct. For example, the breast cancer-specific promoter may comprise a fatty acid synthase or claudin 4 control sequence, whereas the ovarian cancer-specific promoter may comprise a hTERT or survivin control sequence.

Any promoter or control sequence utilized to regulate expression of a therapeutic polynucleotide may utilize specific regulatory sequences that enhance expression and/or post-transcriptional processes, for example. Particular but exemplary sequences include enhancers, a two-step transcriptional amplification system, elements that regulate RNA polyadenylation, half-life, and so forth, such as the WPRE, and/or others in the art.

In other embodiments of the present invention, there are methods of preventing growth of a cell in an individual comprising administering to the individual a construct of the present invention. In specific embodiments, the construct is administered in a liposome and/or the therapeutic gene product may further comprise a protein transduction domain (Schwarze et al., 1999), such as HIV Tat or penetratin, for example. The therapeutic polynucleotide may be administered in a vector such as a plasmid, retroviral vector, adenoviral vector, adeno-associated viral vector, liposome, or a combination thereof, for example.

### I. Nucleic Acid-Based Expression Systems

The present invention utilizes, in some embodiments, systems for expressing therapeutic polynucleotides, particularly for cancer treatment. Particular exemplary aspects for these polynucleotides are described herein.

A. Vectors

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Maniatis et al., 1988 and Ausubel et al., 1994, both incorporated herein by reference.

The term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

1. Promoters and Enhancers

A “promoter” is a control sequence that is a region of a nucleic acid sequence at which initiation and rate of transcription are controlled. In a specific embodiment, a control sequence, such as a promoter, regulates the tissue specificity within which the nucleic acid sequence is expressed. A promoter, or control sequence, may comprise genetic elements at which regulatory proteins and molecules may bind, such as RNA polymerase and other transcription factors, for example. The phrases “operatively positioned,” “operatively linked,” “under control,” and “under transcriptional control” mean that a promoter or other control sequence is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. No. 4,683,202; U.S. Pat. No. 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know the use of promoters, enhancers, and cell type combinations for protein expression, for example, see Sambrook et al. (1989), incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

The identity of tissue-specific promoters or elements, as well as assays to characterize their activity, is well known to those of skill in the art. Tissue-specific promoters utilized to control expression targeting and/or levels of a therapeutic gene product may be comprise wild-type nucleic acid sequence, mutant nucleic acid sequence, or synthetic nucleic acid sequence, so long as the expression of the therapeutic polynucleotide is preferentially retained in one or more tissues of interest compared to tissues that are not the desired target. Control sequences, such as promoters, may be composite sequences, wherein multiple regions are derived from different sources. Synthetic control sequences may be further defined as composite promoters, wherein there are at least two separate regions originating from different endogenous and/or synthetic promoters yet operably linked to control expression of a therapeutic polynucleotide. In a particular embodiment, the tissue specificity refers to specificity for cancerous tissue, as opposed to non-cancerous tissue. The term “cancerous tissue” as used herein refers to a tissue comprising one or more cancer cells.

a. Breast Cancer Tissue-Specific Promoter

Most of the promoters currently used in cancer gene therapy possess strong but unselective activity (e.g. CMV and β-actin promoters) in both normal and tumor cells. Thus, in some aspects of the present invention, a breast tissue-specific promoter is utilized in the invention, such as to control expression of a therapeutic polynucleotide, including a mutant form of Bik, such as the exemplary BikT33D, BikS35D, and Bik T33DS35D mutants (which may be referred to as BikDD), for example. These Bik mutants are described herein but provided in further detail in U.S. Nonprovisional patent application Ser. No. 10/816,698, entitled “Antitumor Effect of Mutant Bik” by Mien-Chie Hung, Yan Li, and Yong Wen, incorporated by reference herein in its entirety. In a particular aspect, the breast cancer-specific promoter of the present invention targets expression of a polynucleotide encoding a therapeutic gene product specifically to breast cancer tissue.

In one particular embodiment of the present invention, composite promoters utilizing either the exemplary fatty acid synthase, muc-1, BCSG1, and/or claudin 4 breast cancer-specific control sequences are employed. The fatty acid synthase or claudin 4 levels are elevated in breast cancer, in specific embodiments, such as would be determined using SAGE analysis and cDNA microarray, for example. In some embodiments, the promoter activity may be enhanced by connecting these two promoters with an enhancer sequence, such as the cytomegalovirus (CMV) promoter enhancer sequence (SEQ ID NO: 1). An exemplary human fatty acid synthase promoter region is provided in SEQ ID NO:6 (National Center for Biotechnology Information GenBank® database Accession No. AF250144). Although SEQ ID NO:7 comprises mRNA for human claudin 4, one of skill in the art recognizes how to obtain the genomic promoter sequence utilizing part or all of this sequence to probe genomic DNA for the adjacent or nearby regulatory sequences. In specific embodiments, the CLDN4 promoter sequence is present in GenBank Accession No. AC093168 (Homo sapiens BAC clone RP11-148M21 from 7, complete sequence).

In specific embodiments, the promoter activity is further enhanced under hypoxic conditions, which usually occur inside solid tumors. To demonstrate its use in cancer gene therapy, one may generate a DNA construct using fatty acid synthase or claudin 4 regulatory regions, for example, to drive apoptotic gene expression. When transfected into cell lines, this construct selectively kills breast cancer cells, in specific embodiments. Moreover, in other specific embodiments, this construct has an anti-tumor effect on breast tumor xenograft in mouse by intravenous injection with an exemplary non-viral delivery system. This indicates that fatty acid synthase or claudin 4 can drive the expression of a therapeutic gene, such as mutant Bik, for example, selectively in breast cancer cells.

In specific embodiments of the breast cancer composite regulatory sequences, the fatty acid synthase and/or claudin 4 sequences are operably linked to other regulatory sequences, such as WPRE, two-step transcriptional amplification (TSTA) system, or both, for example. A skilled artisan recognizes that the term “two-step transcriptional amplification (TSTA) system” may also be referred to as “two-step transcriptional activation (TSTA) system” or “recombinant transcriptional activation approach” (Nettelbeck et al, 2000).

The current invention encompasses breast cancer-specific promoters for control of expression of mutant Bik to target breast cancer cells for treatment that is less toxic or non-toxic to normal tissues.

Specific embodiments to determine whether the promoters had high activity and strict specificity in vivo after systemic delivery, nu/nu nude mice bearing subcutaneous (s.c) or orthotopic (o.t) breast tumor cells may be tail-vein-injected once a day for three consecutive days with the appropriate plasmid DNA-DOTOP:Chol complexes, for example and in vivo and ex vivo bioluminescently images with a non-invasive IVIS™ Imaging System may be obtained. Such images may demonstrate promoter in activity in breast cancer cells and demonstrate that the promoter retains its specificity in vitro and in vivo, thereby providing safer and more effective treatment modalities for breast cancer gene therapy.

b. Ovarian Cancer Tissue-Specific Promoter

Ovarian cancer-specific promoters are useful to target ovarian cancer cells while leaving ovarian non-cancerous cells unaffected. The present inventors developed strong ovarian cancer-specific promoters for targeted expression of polynucleotides encoding therapeutic gene products, including mutant Bik, such as the exemplary BikT33D, BikS35D, and Bik T33DS35D mutants, for example. These Bik mutants are described herein but provided in further detail in U.S. Nonprovisional patent application Ser. No. 10/816,698, entitled “Antitumor Effect of Mutant Bik” by Mien-Chie Hung, Yan Li, and Yong Wen, incorporated by reference herein in its entirety.

In specific aspects of the invention, an ovarian-specific promoter employs hTERT regulatory sequence, ovarian-specific regulatory (OSP1) sequence, ceruloplasmin regulatory sequence, human epididymis protein 4 (He4) regulatory sequence, secretory leukoprotease inhibitor (SLP1) regulatory sequence, and/or survivin regulatory sequence. Exemplary hTERT regulatory sequence may be provided in SEQ ID NO:4, for example. Exemplary survivin regulatory sequence may be provided in Li and Altieri (1999), for example. In particular aspects of the invention, a composite promoter employing a TSTA sequence, such as the exemplary GAL4-VP16 or GAL4-VP2 fusion protein (Iyer et al., 2001; Zhang et al., 2002; Sato et al., 2003; and references cited therein), is utilized to augment the transcriptional activity of the ovarian tissue-specific regulatory sequences. In further embodiments; the post-transcriptional regulatory element of the woodchuck hepatitis virus (WPRE) (SEQ ID NO:2) is utilized to modify RNA polyadenylation signal, RNA export, and/or RNA translation, for example. In a particular aspect, the hTERT-TSTA-WPRE promoter or the survivin-TSTA-WPRE promoter is utilized. Thus, the molecularly engineered promoters are employed for effective treatment modalities for ovarian cancer gene therapy.

Specific embodiments to determine whether these promoters had high activity and strict specificity in vivo after systemic delivery, nu/nu nude mice bearing subcutaneous (s.c) or orthotopic (o.t) ovarian tumor cells may be tail-vein-injected once a day for three consecutive days with the appropriate plasmid DNA-DOTOP:Chol complexes, for example and in vivo and ex vivo bioluminescently images with a non-invasive IVIS™ Imaging System may be obtained. Such images may demonstrate promoter in activity in ovarian cancer cells and demonstrate that the promoter retains its specificity in vitro and in vivo, thereby providing safer and more effective treatment modalities for ovarian cancer gene therapy.

The promoter may also comprise at least the minimal promoter fragment (hTERTp) of the human telomerase reverse transcriptase (hTERT) (SEQ ID NO:4) operably linked to a two-step transcriptional amplification (TSTA) system, such as the exemplary GAL4-VP16 or GAL4-VP2 (two examples of GAL4-VP2 are comprised in SEQ ID NO:3 or SEQ ID NO:5) fusion protein-encoding sequences. The therapeutic polynucleotide may also be operatively linked to a post-transcriptional control sequence, such as the post-transcriptional regulatory element of the woodchuck hepatitis virus (WPRE) to modify RNA polyadenylation signal, RNA export, and/or RNA translation.

Toward an exemplary generation of this promoter, the minimal promoter fragment (hTERTp) of the human telomerase reverse transcriptase (hTERT) (SEQ ID NO:4) may be PCR-amplified from the DNA extracts of LNCaP cells, cells, such as, for example, and tested for activity in luciferase reporter system. A series of composites based on hTERTp promoter then may be engineered by using the GAL4-VP16 or GAL4-VP2 fusion protein through a two-step transcriptional amplification (TSTA) system to augment the transcriptional activity and the post-transcriptional regulatory element of the woodchuck hepatitis virus (WPRE) to modify RNA polyadenylation signal, RNA export, and/or RNA translation. The exemplary GAL4-VP2 fusion protein is encoded by a polynucleotide comprising SEQ ID NO:3 or SEQ ID NO:5.

2. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements. In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, herein incorporated by reference).

3. Multiple Cloning Sites

Vectors can include a multiple cloning site (MCS), which is a nucleic acid region that contains multiple restriction enzyme sites, any of which can be used in conjunction with standard recombinant technology to digest the vector. (See Carbonelli et al., 1999, Levenson et al, 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

4. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression. (See Chandler et al., 1997, herein incorporated by reference.)

5. Polyadenylation Signals

In expression, one will typically include a polyadenylation signal to effect proper polyadenylation of the transcript. The nature of the polyadenylation signal is not believed to be crucial to the successful practice of the invention, and/or any such sequence may be employed. Preferred embodiments include the SV40 polyadenylation signal and/or the bovine growth hormone polyadenylation signal, convenient and/or known to function well in various target cells. Also contemplated as an element of the expression cassette is a transcriptional termination site. These elements can serve to enhance message levels and/or to minimize read through from the cassette into other sequences.

6. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

7. Selectable and Screenable Markers

In certain embodiments of the invention, the cells contain nucleic acid construct of the present invention, a cell may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is calorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

B. Host Cells

The promoters of the present invention may be used in any manner so long as they regulate expression of a particular polynucleotide. Although they are useful for tissue-specific expression, they are by nature promoters/control sequences and, thus, may be used in any cell environment for expressing any polynucleotide.

As used herein, the terms “cell,” “cell line,” and “cell culture” may be used interchangeably. All of these term also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organisms that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny.

Host cells may be derived from prokaryotes or eukaryotes, depending upon whether the desired result is replication of the vector or expression of part or all of the vector-encoded nucleic acid sequences. Numerous cell lines and cultures are available for use as a host cell, and they can be obtained through the American Type Culture Collection (ATCC), which is an organization that serves as an archive for living cultures and genetic materials. An appropriate host can be determined by one of skill in the art based on the vector backbone and the desired result. A plasmid or cosmid, for example, can be introduced into a prokaryote host cell for replication of many vectors. Bacterial cells used as host cells for vector replication and/or expression include DH5α, JM109, and KC8, as well as a number of commercially available bacterial hosts such as SUREOR Competent Cells and Solopack™ Gold Cells (Stratagene®, La Jolla). Alternatively, bacterial cells such as E. coli LE392 could be used as host cells for phage viruses.

Examples of eukaryotic host cells for replication and/or expression of a vector include HeLa, NIH3T3, Jurkat, 293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

C. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available. Although the promoters of the present invention are useful for tissue-specific expression, they are by nature promoters/control sequences and, thus, may be used in any expression system so long as they regulate expression of a particular polynucleotide.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MaxBac® 2.0 from Invitrogen® and BacPack™ Baculovirus Expression System From Clontech®.

Other examples of expression systems include Stratagene®'s Complete Control™ Inducible Mammalian Expression System, which involves a synthetic ecdysone-inducible receptor, or its pET Expression System, an E. coli expression system. Another example of an inducible expression system is available from Invitrogen®, which carries the T-Rex™ (tetracycline-regulated expression) System, an inducible mammalian expression system that uses the full-length CMV promoter. Invitrogen® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

### II. Nucleic Acid Compositions

In certain embodiments of the present invention, particular sequences are employed in the inventive polynucleotide constructs and uses thereof. Although a skilled artisan recognizes that these specific sequences may be employed exactly as provided herein, in other embodiments sequences that are similar to those exemplary sequences provided herein are useful at least in part for tissue-specific cancer regulatory sequences.

Certain embodiments of the present invention concern a tissue-specific regulatory nucleic acid (nucleic acid may interchangeably be used with the term “polynucleotide”). In other aspects, an expression construct nucleic acid comprises a nucleic acid segment of the exemplary SEQ ID NO:1, SEQ ID NO:2, SEQ ID NO:3, SEQ ID NO:4, SEQ ID NO:5, SEQ ID NO:6, SEQ ID NO:7, or a biologically functional equivalent thereof.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompass the terms “oligonucleotide” and “polynucleotide,” each as a subgenus of the term “nucleic acid.” The term “oligonucleotide” refers to a molecule of between about 3 and about 100 nucleobases in length. The term “polynucleotide” refers to at least one molecule of greater than about 100 nucleobases in length.

These definitions generally refer to a single-stranded molecule, but in specific embodiments will also encompass an additional strand that is partially, substantially or fully complementary to the single-stranded molecule. Thus, a nucleic acid may encompass a double-stranded molecule or a triple-stranded molecule that comprises one or more complementary strand(s) or “complement(s)” of a particular sequence comprising a molecule. As used herein, a single stranded nucleic acid may be denoted by the prefix “ss,” a double stranded nucleic acid by the prefix “ds,” and a triple stranded nucleic acid by the prefix “ts.”

A. Nucleobases

As used herein a “nucleobase” refers to a heterocyclic base, such as for example a naturally occurring nucleobase (i.e., an A, T, G, C or U) found in at least one naturally occurring nucleic acid (i.e., DNA and RNA), and naturally or non-naturally occurring derivative(s) and analogs of such a nucleobase. A nucleobase generally can form one or more hydrogen bonds (“anneal” or “hybridize”) with at least one naturally occurring nucleobase in manner that may substitute for naturally occurring nucleobase pairing (e.g., the hydrogen bonding between A and T, G and C, and A and U).

“Purine” and/or “pyrimidine” nucleobase(s) encompass naturally occurring purine and/or pyrimidine nucleobases and also derivative(s) and analog(s) thereof, including but not limited to, those a purine or pyrimidine substituted by one or more of an alkyl, caboxyalkyl, amino, hydroxyl, halogen (i.e., fluoro, chloro, bromo, or iodo), thiol or alkylthiol moeity. Preferred alkyl (e.g., alkyl, caboxyalkyl, etc.) moeities comprise of from about 1, about 2, about 3, about 4, about 5, to about 6 carbon atoms. Other non-limiting examples of a purine or pyrimidine include a deazapurine, a 2,6-diaminopurine, a 5-fluorouracil, a xanthine, a hypoxanthine, a 8-bromoguanine, a 8-chloroguanine, a bromothymine, a 8-aminoguanine, a 8-hydroxyguanine, a 8-methylguanine, a 8-thioguanine, an azaguanine, a 2-aminopurine, a 5-ethylcytosine, a 5-methylcyosine, a 5-bromouracil, a 5-ethyluracil, a 5-iodouracil, a 5-chlorouracil, a 5-propyluracil, a thiouracil, a 2-methyladenine, a methylthioadenine, a N,N-diemethyladenine, an azaadenines, a 8-bromoadenine, a 8-hydroxyadenine, a 6-hydroxyaminopurine, a 6-thiopurine, a 4-(6-aminohexyl/cytosine), and the like.

A nucleobase may be comprised in a nucleside or nucleotide, using any chemical or natural synthesis method described herein or known to one of ordinary skill in the art.

B. Nucleosides

As used herein, a “nucleoside” refers to an individual chemical unit comprising a nucleobase covalently attached to a nucleobase linker moiety. A non-limiting example of a “nucleobase linker moiety” is a sugar comprising 5-carbon atoms (i.e., a “5-carbon sugar”), including but not limited to a deoxyribose, a ribose, an arabinose, or a derivative or an analog of a 5-carbon sugar. Non-limiting examples of a derivative or an analog of a 5-carbon sugar include a 2′-fluoro-2′-deoxyribose or a carbocyclic sugar where a carbon is substituted for an oxygen atom in the sugar ring.

Different types of covalent attachment(s) of a nucleobase to a nucleobase linker moiety are known in the art. By way of non-limiting example, a nucleoside comprising a purine (i.e., A or G) or a 7-deazapurine nucleobase typically covalently attaches the 9 position of a purine or a 7-deazapurine to the 1′-position of a 5-carbon sugar. In another non-limiting example, a nucleoside comprising a pyrimidine nucleobase (i.e., C, T or U) typically covalently attaches a 1 position of a pyrimidine to a 1′-position of a 5-carbon sugar (Kornberg and Baker, 1992).

C. Nucleotides

As used herein, a “nucleotide” refers to a nucleoside further comprising a “backbone moiety”. A backbone moiety generally covalently attaches a nucleotide to another molecule comprising a nucleotide, or to another nucleotide to form a nucleic acid. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when a nucleotide comprises derivatives or analogs of a naturally occurring 5-carbon sugar or phosphorus moiety.

D. Nucleic Acid Analogs

A nucleic acid may comprise, or be composed entirely of, a derivative or analog of a nucleobase, a nucleobase linker moiety and/or backbone moiety that may be present in a naturally occurring nucleic acid. As used herein a “derivative” refers to a chemically modified or altered form of a naturally occurring molecule, while the terms “mimic” or “analog” refer to a molecule that may or may not structurally resemble a naturally occurring molecule or moiety, but possesses similar functions. As used herein, a “moiety” generally refers to a smaller chemical or molecular component of a larger chemical or molecular structure. Nucleobase, nucleoside and nucleotide analogs or derivatives are well known in the art, and have been described (see for example, Scheit, 1980, incorporated herein by reference).

Additional non-limiting examples of nucleosides, nucleotides or nucleic acids comprising 5-carbon sugar and/or backbone moiety derivatives or analogs, include those in U.S. Pat. No. 5,681,947 which describes oligonucleotides comprising purine derivatives that form triple helixes with and/or prevent expression of dsDNA; U.S. Pat. Nos. 5,652,099 and 5,763,167 which describe nucleic acids incorporating fluorescent analogs of nucleosides found in DNA or RNA, particularly for use as fluorescent nucleic acids probes; U.S. Pat. No. 5,614,617 which describes oligonucleotide analogs with substitutions on pyrimidine rings that possess enhanced nuclease stability; U.S. Pat. Nos. 5,670,663, 5,872,232 and 5,859,221 which describe oligonucleotide analogs with modified 5-carbon sugars (i.e., modified 2′-deoxyfuranosyl moieties) used in nucleic acid detection; U.S. Pat. No. 5,446,137 which describes oligonucleotides comprising at least one 5-carbon sugar moiety substituted at the 4′ position with a substituent other than hydrogen that can be used in hybridization assays; U.S. Pat. No. 5,886,165 which describes oligonucleotides with both deoxyribonucleotides with 3′-5′ internucleotide linkages and ribonucleotides with 2′-5′ internucleotide linkages; U.S. Pat. No. 5,714,606 which describes a modified internucleotide linkage wherein a 3′-position oxygen of the internucleotide linkage is replaced by a carbon to enhance the nuclease resistance of nucleic acids; U.S. Pat. No. 5,672,697 which describes oligonucleotides containing one or more 5′ methylene phosphonate internucleotide linkages that enhance nuclease resistance; U.S. Pat. Nos. 5,466,786 and 5,792,847 which describe the linkage of a substituent moeity which may comprise a drug or label to the 2′ carbon of an oligonucleotide to provide enhanced nuclease stability and ability to deliver drugs or detection moieties; U.S. Pat. No. 5,223,618 which describes oligonucleotide analogs with a 2 or 3 carbon backbone linkage attaching the 4′ position and 3′ position of adjacent 5-carbon sugar moiety to enhanced cellular uptake, resistance to nucleases and hybridization to target RNA; U.S. Pat. No. 5,470,967 which describes oligonucleotides comprising at least one sulfamate or sulfamide internucleotide linkage that are useful as nucleic acid hybridization probe; U.S. Pat. Nos. 5,378,825, 5,777,092, 5,623,070, 5,610,289 and 5,602,240 which describe oligonucleotides with three or four atom linker moeity replacing phosphodiester backbone moeity used for improved nuclease resistance, cellular uptake and regulating RNA expression; U.S. Pat. No. 5,858,988 which describes hydrophobic carrier agent attached to the 2′-O position of oligonucleotides to enhanced their membrane permeability and stability; U.S. Pat. No. 5,214,136 which describes oligonucleotides conjugated to anthraquinone at the 5′ terminus that possess enhanced hybridization to DNA or RNA; enhanced stability to nucleases; U.S. Pat. No. 5,700,922 which describes PNA-DNA-PNA chimeras wherein the DNA comprises 2′-deoxy-erythro-pentofuranosyl nucleotides for enhanced nuclease resistance, binding affinity, and ability to activate RNase H; and U.S. Pat. No. 5,708,154 which describes RNA linked to a DNA to form a DNA-RNA hybrid.

E. Preparation of Nucleic Acids

A nucleic acid may be made by any technique known to one of ordinary skill in the art, such as for example, chemical synthesis, enzymatic production or biological production. Non-limiting examples of a synthetic nucleic acid (e.g., a synthetic oligonucleotide), include a nucleic acid made by in vitro chemically synthesis using phosphotriester, phosphite or phosphoramidite chemistry and solid phase techniques such as described in EP 266,032, incorporated herein by reference, or via deoxynucleoside H-phosphonate intermediates as described by Froehler et al., 1986 and U.S. Pat. No. 5,705,629, each incorporated herein by reference. In the methods of the present invention, one or more oligonucleotide may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. No. 4,683,202 and U.S. Pat. No. 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 1989, incorporated herein by reference).

F. Purification of Nucleic Acids

A nucleic acid may be purified on polyacrylamide gels, cesium chloride centrifugation gradients, or by any other means known to one of ordinary skill in the art (see for example, Sambrook et al., 1989, incorporated herein by reference).

In certain aspect, the present invention concerns a nucleic acid that is an isolated nucleic acid. As used herein, the term “isolated nucleic acid” refers to a nucleic acid molecule (e.g., an RNA or DNA molecule) that has been isolated free of, or is otherwise free of, the bulk of the total genomic and transcribed nucleic acids of one or more cells. In certain embodiments, “isolated nucleic acid” refers to a nucleic acid that has been isolated free of, or is otherwise free of, bulk of cellular components or in vitro reaction components such as for example, macromolecules such as lipids or proteins, small biological molecules, and the like.

G. Nucleic Acid Segments

In certain embodiments, the nucleic acid is a nucleic acid segment. As used herein, the term “nucleic acid segment,” are smaller fragments of a nucleic acid, such as for non-limiting example, those that comprise only part of the regulatory sequences for a given transcribed polynucleotide.

H. Nucleic Acid Complements

The present invention also encompasses a nucleic acid that is complementary to a nucleic acid of the invention. In particular embodiments the invention encompasses a nucleic acid or a nucleic acid segment complementary to the sequence set forth in SEQ ID NO: 1, SEQ ID NO:2, SEQ ID NO:3, SEQ ID NO:4 and SEQ ID NO:5, for example. A nucleic acid is “complement(s)” or is “complementary” to another nucleic acid when it is capable of base-pairing with another nucleic acid according to the standard Watson-Crick, Hoogsteen or reverse Hoogsteen binding complementarity rules. As used herein “another nucleic acid” may refer to a separate molecule or a spatial separated sequence of the same molecule.

As used herein, the term “complementary” or “complement(s)” also refers to a nucleic acid comprising a sequence of consecutive nucleobases or semiconsecutive nucleobases (e.g., one or more nucleobase moieties are not present in the molecule) capable of hybridizing to another nucleic acid strand or duplex even if less than all the nucleobases do not base pair with a counterpart nucleobase. In certain embodiments, a “complementary” nucleic acid comprises a sequence in which about 70%, about 71%, about 72%, about 73%, about 74%, about 75%, about 76%, about 77%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range derivable therein, of the nucleobase sequence is capable of base-pairing with a single or double stranded nucleic acid molecule during hybridization. In certain embodiments, the term “complementary” refers to a nucleic acid that may hybridize to another nucleic acid strand or duplex in stringent conditions, as would be understood by one of ordinary skill in the art.

In certain embodiments, a “partly complementary” nucleic acid comprises a sequence that may hybridize in low stringency conditions to a single or double stranded nucleic acid, or contains a sequence in which less than about 70% of the nucleobase sequence is capable of base-pairing with a single or double stranded nucleic acid molecule during hybridization.

I. Hybridization

As used herein, “hybridization”, “hybridizes” or “capable of hybridizing” is understood to mean the forming of a double or triple stranded molecule or a molecule with partial double or triple stranded nature. The term “anneal” as used herein is synonymous with “hybridize.” The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those conditions that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. Such conditions are termed “low stringency” or “low stringency conditions”, and non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suite a particular application.

The nucleic acid(s) of the present invention, regardless of the length of the sequence itself, may be combined with other nucleic acid sequences, including but not limited to, promoters, enhancers, polyadenylation signals, restriction enzyme sites, multiple cloning sites, coding segments, and the like, to create one or more nucleic acid construct(s). As used herein, a “nucleic acid construct” is a nucleic acid engineered or altered by the hand of man, and generally comprises one or more nucleic acid sequences organized by the hand of man.

In a non-limiting example, one or more nucleic acid constructs may be prepared that include a contiguous stretch of nucleotides identical to or complementary to promoter sequences of the invention, for example. A nucleic acid construct may be about 3, about 5, about 8, about 10 to about 14, or about 15, about 20, about 30, about 40, about 50, about 100, about 200, about 500, about 1,000, about 2,000, about 3,000, about 5,000, about 10,000, about 15,000, about 20,000, about 30,000, about 50,000, about 100,000, about 250,000, about 500,000, about 750,000, to about 1,000,000 nucleotides in length, as well as constructs of greater size, up to and including chromosomal sizes (including all intermediate lengths and intermediate ranges), given the advent of nucleic acids constructs such as a yeast artificial chromosome are known to those of ordinary skill in the art. It will be readily understood that “intermediate lengths” and “intermediate ranges”, as used herein, means any length or range including or between the quoted values (i.e., all integers including and between such values). Non-limiting examples of intermediate lengths include about 11, about 12, about 13, about 16, about 17, about 18, about 19, etc.; about 21, about 22, about 23, etc.; about 31, about 32, etc.; about 51, about 52, about 53, etc.; about 101, about 102, about 103, etc.; about 151, about 152, about 153, etc.; about 1,001, about 1002, etc.; about 50,001, about 50,002, etc; about 750,001, about 750,002, etc.; about 1,000,001, about 1,000,002, etc. Non-limiting examples of intermediate ranges include about 3 to about 32, about 150 to about 500,001, about 3,032 to about 7,145, about 5,000 to about 15,000, about 20,007 to about 1,000,003, etc.

The term “a sequence essentially as set forth in SEQ ID NO:4”, for example, means that the sequence substantially corresponds to a portion of SEQ ID NO:4 and has relatively few nucleotides that are not identical to, or a biologically functional equivalent of, the nucleotides of SEQ ID NO:4. Thus, “a sequence essentially as set forth in SEQ ID NO:4” encompasses nucleic acids, nucleic acid segments, and genes that comprise part or all of the nucleic acid sequences as set forth in SEQ ID NO:4. SEQ ID NO:4 is referred to herein solely as an illustrative embodiment, and one of skill in the art recognizes that such description analogously applies to other specific sequences of the invention.

The term “biologically functional equivalent” is well understood in the art and is further defined in detail herein. Accordingly, a sequence that has between about 70% and about 80%; or more preferably, between about 81% and about 90%; or even more preferably, between about 91% and about 99%; of nucleotides that are identical or functionally equivalent to the nucleotides of sequences referred to herein, such as the exemplary SEQ ID NO:1, SEQ ID NO:2, SEQ ID NO:3, SEQ ID NO:4, SEQ ID NO:5, or SEQ ID NO:6 will be a sequence that is respectively “essentially as set forth in the SEQ ID NO:1, SEQ ID NO:2, SEQ ID NO:3, SEQ ID NO:4, SEQ ID NO:5, or SEQ ID NO:6”, provided the biological activity of the sequences is maintained.

In certain other embodiments, the invention concerns at least one recombinant vector that include within its sequence a nucleic acid sequence essentially as set forth in SEQ ID NO: 1, SEQ ID NO:2, SEQ ID NO:3, SEQ ID NO:4 or SEQ ID NO:5.

### III. Therapeutic Polynucleotides

The therapeutic polynucleotide which expression is controlled by the inventive control sequences encompassed by the invention may be of any kind, so long as the gene product encoded thereby generates an anticancer effect. Anticancer effects include inducing apoptosis in at least one cancer cell, inhibiting proliferation of at least one cancer cell, ameliorating at least once symptom of cancer in an individual, and so forth. In particular embodiments, the therapeutic polynucleotide encodes a mutant form of Bik, including the exemplary BikT33D, BikS35D, and Bik T33DS35D mutants, for example, which are described in U.S. patent application Ser. No. 10/816,698, incorporated by reference herein in its entirety. In an exemplary case, EIA is employed as the therapeutic polynucleotide, and exemplary EIA polynucleotides are provided in U.S. Pat. No. 7,005,424 and U.S. Pat. No. 6,683,059, both of which are incorporated by reference herein in their entirety.

The therapeutic polynucleotide may be of any kind known to those of skill in the art or discovered later. In particular embodiments, they encode inhibitors of cellular proliferation, regulators of programmed cell death, tumor suppressors and/or antisense sequences of inducers of cellular proliferation. The therapeutic polynucleotide may encode small interfering RNAs or antisense sequences. Examples of therapeutic polynucleotides include those encoding TNFα or p53 or that encode polypeptide inducers of apoptosis including, but not limited to, Bik, p53, Bax, Bak, Bcl-x, Bad, Bim, Bok, Bid, Harakiri, Ad E1B, Bad and ICE-CED3 proteases. Other exemplary therapeutic polynucleotides include those that encode retinoblastoma, Blk, IL-12, IL-10, IFN-a, cytosine deaminase, GM-CSF, E1A, and other pro-apoptotic proteins, for example. A polynucleotide encoding an amino acid substitution at threonine 33, serine 35, or both of mutant Bik may be utilized. In particular aspects of these embodiments, the amino acids of the mutant Bik polypeptide are substituted with aspartate. In other particular aspects, one or more phosphorylation sites are defective in a mutant Bik. Additional therapeutic polynucleotides include TNFα or p53 or inducers of apoptosis including, but not limited to, Bik, p53, Bax, Bak, Bcl-x, Bad, Bim, Bok, Bid, Harakiri, Ad E1B, Bad and ICE-CED3 proteases.

### IV. Nucleic Acid Delivery

The general approach to the aspects of the present invention concerning compositions and/or therapeutics is to provide a cell with a gene construct encoding a specific and/or desired mutant Bik protein, polypeptide, or peptide, thereby permitting the desired activity of the protein, polypeptide, or peptide to take effect. While it is conceivable that the gene construct and/or protein may be delivered directly, a preferred embodiment involves providing a nucleic acid encoding a specific and desired protein, polypeptide, or peptide to the cell. Following this provision, the proteinaceous composition is synthesized by the transcriptional and translational machinery of the cell, as well as any that may be provided by the expression construct. In providing antisense, ribozymes and other inhibitors, the preferred mode is also to provide a nucleic acid encoding the construct to the cell.

In certain embodiments of the invention, the nucleic acid encoding the gene may be stably integrated into the genome of the cell. In yet further embodiments, the nucleic acid may be stably maintained in the cell as a separate, episomal segment of DNA. Such nucleic acid segments and “episomes” encode sequences sufficient to permit maintenance and replication independent of and in synchronization with the host cell cycle. How the expression construct is delivered to a cell and/or where in the cell the nucleic acid remains is dependent on the type of expression construct employed.

A. DNA Delivery Using Viral Vectors

The ability of certain viruses to infect cells and enter cells via receptor-mediated endocytosis, and to integrate into host cell genome and/or express viral genes stably and/or efficiently have made them attractive candidates for the transfer of foreign genes into mammalian cells. Preferred gene therapy vectors of the present invention will generally be viral vectors.

Although some viruses that can accept foreign genetic material are limited in the number of nucleotides they can accommodate and/or in the range of cells they infect, these viruses have been demonstrated to successfully effect gene expression. However, adenoviruses do not integrate their genetic material into the host genome and/or therefore do not require host replication for gene expression, making them ideally suited for rapid, efficient, heterologous gene expression. Techniques for preparing replication-defective infective viruses are well known in the art.

Of course, in using viral delivery systems, one will desire to purify the virion sufficiently to render it essentially free of undesirable contaminants, such as defective interfering viral particles and endotoxins and other pyrogens such that it will not cause any untoward reactions in the cell, animal and/or individual receiving the vector construct. A preferred means of purifying the vector involves the use of buoyant density gradients, such as cesium chloride gradient centrifugation.

1. Adenoviral Vectors

A particular method for delivery of the expression constructs involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and/or (b) to ultimately express a tissue and/or cell-specific construct that has been cloned therein.

The expression vector comprises a genetically engineered form of adenovirus. Knowledge of the genetic organization and adenovirus, a 36 kb, linear, double-stranded DNA virus, allows substitution of large pieces of adenoviral DNA with foreign sequences up to 7 kb (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and/or no genome rearrangement has been detected after extensive amplification.

Adenovirus is particularly suitable for use as a gene transfer vector because of its mid-sized genome, ease of manipulation, high titer, wide target-cell range and/or high infectivity. Both ends of the viral genome contain 100-200 base pair inverted repeats (ITRs), which are cis elements necessary for viral DNA replication and/or packaging. The early (E) and/or late (L) regions of the genome contain different transcription units that are divided by the onset of viral DNA replication. The E1 region (EIA and/or E1B) encodes proteins responsible for the regulation of transcription of the viral genome and/or a few cellular genes. The expression of the E2 region (E2A and/or E2B) results in the synthesis of the proteins for viral DNA replication. These proteins are involved in DNA replication, late gene expression and/or host cell shut-off (Renan, 1990). The products of the late genes, including the majority of the viral capsid proteins, are expressed only after significant processing of a single primary transcript issued by the major late promoter (MLP). The MLP (located at 16.8 m.u.) is particularly efficient during the late phase of infection, and/or all the mRNA's issued from this promoter possess a 5′-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and/or examine its genomic structure.

Generation and/or propagation of the current adenovirus vectors, which are replication deficient, depend on a unique helper cell line, designated 293, which was transformed from human embryonic kidney cells by Ad5 DNA fragments and constitutively expresses E1 proteins (E1A and/or E1B; Graham et al., 1977). Since the E3 region is dispensable from the adenovirus genome (Jones and Shenk, 1978), the current adenovirus vectors, with the help of 293 cells, carry foreign DNA in either the E1, the D3 and both regions (Graham and Prevec, 1991). Recently, adenoviral vectors comprising deletions in the E4 region have been described (U.S. Pat. No. 5,670,488, incorporated herein by reference).

In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and/or E3 regions, the maximum capacity of the current adenovirus vector is under 7.5 kb, and/or about 15% of the total length of the vector. More than 80% of the adenovirus viral genome remains in the vector backbone.

Helper cell lines may be derived from human cells such as human embryonic kidney cells, muscle cells, hematopoietic cells and other human embryonic mesenchymal and epithelial cells. Alternatively, the helper cells may be derived from the cells of other mammalian species that are permissive for human adenovirus. Such cells include, e.g. Vero cells and other monkey embryonic mesenchymal and/or epithelial cells. As stated above, the preferred helper cell line is 293.

Recently, Racher et al. (1995) disclosed improved methods for culturing 293 cells and/or propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and/or left stationary, with occasional agitation, for 1 to 4 h. The medium is then replaced with 50 ml of fresh medium and/or shaking initiated. For virus production, cells are allowed to grow to about 80% confluence, after which time the medium is replaced (to 25% of the final volume) and/or adenovirus added at an MOI of 0.05. Cultures are left stationary overnight, following which the volume is increased to 100% and/or shaking commenced for another 72 h.

Other than the requirement that the adenovirus vector be replication defective, and at least conditionally defective, the nature of the adenovirus vector is not believed to be crucial to the successful practice of the invention. The adenovirus may be of any of the 42 different known serotypes and subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the transforming construct at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al. (1986) and in the E4 region where a helper cell line and helper virus complements the E4 defect.

Adenovirus growth and/or manipulation is known to those of skill in the art, and/or exhibits broad host range in vitro and in vivo. This group of viruses can be obtained in high titers, e.g., 10⁹ to 10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and/or therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1992). Recently, animal studies suggested that recombinant adenovirus could be used for gene therapy (Stratford-Perricaudet and Perricaudet, 1991a; Stratford-Perricaudet et al., 1991b; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and/or stereotactic inoculation into the brain (Le Gal La Salle et al., 1993). Recombinant adenovirus and adeno-associated virus (see below) can both infect and transduce non-dividing human primary cells.

2. AAV Vectors

Adeno-associated virus (AAV) is an attractive vector system for use in the cell transduction of the present invention as it has a high frequency of integration and it can infect nondividing cells, thus making it useful for delivery of genes into mammalian cells, for example, in tissue culture (Muzyczka, 1992) and in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. No. 5,139,941 and/or U.S. Pat. No. 4,797,368, each incorporated herein by reference.

Studies demonstrating the use of AAV in gene delivery include LaFace et al. (1988); Zhou et al. (1993); Flotte et al. (1993); and Walsh et al. (1994). Recombinant AAV vectors have been used successfully for in vitro and/or in vivo transduction of marker genes (Kaplitt et al., 1994; Lebkowski et al., 1988; Samulski et al., 1989; Yoder et al., 1994; Zhou et al., 1994; Hermonat and Muzyczka, 1984; Tratschin et al., 1985; McLaughlin et al., 1988) and genes involved in human diseases (Flotte et al., 1992; Luo et al., 1994; Ohi et al., 1990; Walsh et al., 1994; Wei et al., 1994). Recently, an AAV vector has been approved for phase I human trials for the treatment of cystic fibrosis.

AAV is a dependent parvovirus in that it requires coinfection with another virus (either adenovirus and a member of the herpes virus family) to undergo a productive infection in cultured cells (Muzyczka, 1992). In the absence of coinfection with helper virus, the wild type AAV genome integrates through its ends into human chromosome 19 where it resides in a latent state as a provirus (Kotin et al., 1990; Samulski et al., 1991). rAAV, however, is not restricted to chromosome 19 for integration unless the AAV Rep protein is also expressed (Shelling and Smith, 1994). When a cell carrying an AAV provirus is superinfected with a helper virus, the AAV genome is “rescued” from the chromosome and from a recombinant plasmid, and/or a normal productive infection is established (Samulski et al., 1989; McLaughlin et al., 1988; Kotin et al., 1990; Muzyczka, 1992).

Typically, recombinant AAV (rAAV) virus is made by cotransfecting a plasmid containing the gene of interest flanked by the two AAV terminal repeats (McLaughlin et al., 1988; Samulski et al., 1989; each incorporated herein by reference) and/or an expression plasmid containing the wild type AAV coding sequences without the terminal repeats, for example pIM45 (McCarty et al., 1991; incorporated herein by reference). The cells are also infected and transfected with adenovirus and plasmids carrying the adenovirus genes required for AAV helper function. rAAV virus stocks made in such fashion are contaminated with adenovirus which must be physically separated from the rAAV particles (for example, by cesium chloride density centrifugation). Alternatively, adenovirus vectors containing the AAV coding regions and cell lines containing the AAV coding regions and some and all of the adenovirus helper genes could be used (Yang et al., 1994; Clark et al., 1995). Cell lines carrying the rAAV DNA as an integrated provirus can also be used (Flotte et al., 1995).

3. Retroviral Vectors

Retroviruses have promise as gene delivery vectors due to their ability to integrate their genes into the host genome, transferring a large amount of foreign genetic material, infecting a broad spectrum of species and cell types and of being packaged in special cell-lines (Miller, 1992).

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and/or directs synthesis of viral proteins. The integration results in the retention of the viral gene sequences in the recipient cell and/or its descendants. The retroviral genome contains three genes, gag, pol, and/or env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and/or stable expression require the division of host cells (Paskind et al., 1975).

Concern with the use of defective retrovirus vectors is the potential appearance of wild-type replication-competent virus in the packaging cells. This can result from recombination events in which the intact sequence from the recombinant virus inserts upstream from the gag, pol, env sequence integrated in the host cell genome. However, new packaging cell lines are now available that should greatly decrease the likelihood of recombination (Markowitz et al, 1988; Hersdorffer et al, 1990).

Gene delivery using second generation retroviral vectors has been reported. Kasahara et al. (1994) prepared an engineered variant of the Moloney murine leukemia virus, that normally infects only mouse cells, and modified an envelope protein so that the virus specifically bound to, and infected, human cells bearing the erythropoietin (EPO) receptor. This was achieved by inserting a portion of the EPO sequence into an envelope protein to create a chimeric protein with a new binding specificity.

4. Other Viral Vectors

Other viral vectors may be employed as expression constructs in the present invention. Vectors derived from viruses such as vaccinia virus (Ridgeway, 1988; Baichwal and Sugden, 1986; Coupar et al., 1988), sindbis virus, cytomegalovirus and/or herpes simplex virus may be employed. They offer several attractive features for various mammalian cells (Friedmann, 1989; Ridgeway, 1988; Baichwal and Sugden, 1986; Coupar et al., 1988; Horwich et al., 1990).

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. Chang et al. recently introduced the chloramphenicol acetyltransferase (CAT) gene into duck hepatitis B virus genome in the place of the polymerase, surface, and/or pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

In certain further embodiments, the gene therapy vector will be HSV. A factor that makes HSV an attractive vector is the size and organization of the genome. Because HSV is large, incorporation of multiple genes and expression cassettes is less problematic than in other smaller viral systems. In addition, the availability of different viral control sequences with varying performance (temporal, strength, etc.) makes it possible to control expression to a greater extent than in other systems. It also is an advantage that the virus has relatively few spliced messages, further easing genetic manipulations. HSV also is relatively easy to manipulate and/or can be grown to high titers. Thus, delivery is less of a problem, both in terms of volumes needed to attain sufficient MOI and in a lessened need for repeat dosings.

5. Modified Viruses

In still further embodiments of the present invention, the nucleic acids to be delivered are housed within an infective virus that has been engineered to express a specific binding ligand. The virus particle will thus bind specifically to the cognate receptors of the target cell and deliver the contents to the cell. A novel approach designed to allow specific targeting of retrovirus vectors was recently developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and/or against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

B. Other Methods of DNA Delivery

In various embodiments of the invention, DNA is delivered to a cell as an expression construct. In order to effect expression of a gene construct, the expression construct must be delivered into a cell. As described herein, the preferred mechanism for delivery is via viral infection, where the expression construct is encapsidated in an infectious viral particle. However, several non-viral methods for the transfer of expression constructs into cells also are contemplated by the present invention. In one embodiment of the present invention, the expression construct may consist only of naked recombinant DNA and/or plasmids. Transfer of the construct may be performed by any of the methods mentioned which physically and/or chemically permeabilize the cell membrane. Some of these techniques may be successfully adapted for in vivo and/or ex vivo use, as discussed below.

C. Liposome-Mediated Transfection

In a further embodiment of the invention, the expression construct may be entrapped in a liposome. Liposomes are vesicular structures characterized by a phospholipid bilayer membrane and/or an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and/or entrap water and/or dissolved solutes between the lipid bilayers (Ghosh and Bachhawat, 1991). Also contemplated is an expression construct complexed with Lipofectamine (Gibco BRL).

Liposome-mediated nucleic acid delivery and expression of foreign DNA in vitro has been very successful (Nicolau and Sene, 1982; Fraley et al., 1979; Nicolau et al., 1987). Wong et al. (1980) demonstrated the feasibility of liposome-mediated delivery and/or expression of foreign DNA in cultured chick embryo, HeLa and hepatoma cells.

In certain embodiments of the invention, the liposome may be complexed with a hemagglutinating virus (HVJ). This has been shown to facilitate fusion with the cell membrane and/or promote cell entry of liposome-encapsulated DNA (Kaneda et al., 1989). In other embodiments, the liposome may be complexed and/or employed in conjunction with nuclear non-histone chromosomal proteins (HMG-1) (Kato et al., 1991). In yet further embodiments, the liposome may be complexed and/or employed in conjunction with both HVJ and HMG-1. In other embodiments, the delivery vehicle may comprise a ligand and a liposome. Where a bacterial promoter is employed in the DNA construct, it also will be desirable to include within the liposome an appropriate bacterial polymerase.

The inventors contemplate that neu-suppressing gene products can be introduced into cells using liposome-mediated gene transfer. It is proposed that such constructs can be coupled with liposomes and directly introduced via a catheter, as described by Nabel et al. (1990). By employing these methods, the neu-suppressing gene products can be expressed efficiently at a specific site in vivo, not just the liver and spleen cells which are accessible via intravenous injection. Therefore, this invention also encompasses compositions of DNA constructs encoding a neu-suppressing gene product formulated as a DNA/liposome complex and methods of using such constructs.

As described in U.S. Pat. No. 5,641,484, liposomes are particularly well suited for the treatment of HER2/neu-mediated cancer.

Catatonic liposomes that are efficient transfection reagents for Bik for animal cells can be prepared using the method of Gao et al. (1991). Gao et al. describes a novel catatonic cholesterol derivative that can be synthesized in a single step. Liposomes made of this lipid are reportedly more efficient in transfection and less toxic to treated cells than those made with the reagent Lipofectin. These lipids are a mixture of DC-Chol (“3□(N—(N′N′-dimethylaminoethane)-carbamoyl cholesterol”) and DOPE (“dioleoylphosphatidylethanolamine”). The steps in producing these liposomes are as follows.

DC-Chol is synthesized by a simple reaction from cholesteryl chloroformate and N,N-Dimethylethylenediamine. A solution of cholesteryl chloroformate (2.25 g, 5 mmol in 5 ml dry chloroform) is added dropwise to a solution of excess N,N-Dimethylethylenediamine (2 ml, 18.2 mmol in 3 ml dry chloroform) at 0° C. Following removal of the solvent by evaporation, the residue is purified by recrystallization in absolute ethanol at 4° C. and dried in vacuo. The yield is a white powder of DC-Chol.

Cationic liposomes are prepared by mixing 1.2 μmol of DC-Chol and 8.0 μmol of DOPE in chloroform. This mixture is then dried, vacuum desiccated, and resuspended in 1 ml sterol 20 mM Hepes buffer (pH 7.8) in a tube. After 24 hours of hydration at 4° C., the dispersion is sonicated for 5-10 minutes in a sonicator form liposomes with an average diameter of 150-200 nm.

To prepare a liposome/DNA complex, the inventors use the following steps. The DNA to be transfected is placed in DMEM/F12 medium in a ratio of 15 μg DNA to 50 μl DMEM/F12. DMEM/F12 is then used to dilute the DC-Chol/DOPE liposome mixture to a ratio of 50 μl DMEM/F12 to 100 μl liposome. The DNA dilution and the liposome dilution are then gently mixed, and incubated at 37° C. for 10 minutes. Following incubation, the DNA/liposome complex is ready for injection.

Liposomal transfection can be via liposomes composed of, for example, phosphatidylcholine (PC), phosphatidylserine (PS), cholesterol (Chol), N-[1-(2,3-dioleyloxy)propyl]-N,N-trimethylammonium chloride (DOTMA), dioleoylphosphatidylethanolamine (DOPE), and/or 3 beta.[N—(N′N′-dimethylaminoethane)-carbamoyl cholesterol (DC-Chol), as well as other lipids known to those of skill in the alt. Those of skill in the art will recognize that there are a variety of liposomal transfection techniques that will be useful in the present invention. Among these techniques are those described in Nicolau et al., 1987, Nabel et al, 1990, and Gao et al., 1991. In a specific embodiment, the liposomes comprise DC-Chol. More particularly, the inventors the liposomes comprise DC-Chol and DOPE that have been prepared following the teaching of Gao et al. (1991) in the manner described in the Preferred Embodiments Section. The inventors also anticipate utility for liposomes comprised of DOTMA, such as those that are available commercially under the trademark Lipofectin™, from Vical, Inc., in San Diego, Calif.

Liposomes may be introduced into contact with cells to be transfected by a variety of methods. In cell culture, the liposome-DNA complex can simply be dispersed in the cell culture solution. For application in vivo, liposome-DNA complex are typically injected. Intravenous injection allow liposome-mediated transfer of DNA complex, for example, the liver and the spleen. In order to allow transfection of DNA into cells that are not accessible through intravenous injection, it is possible to directly inject the liposome-DNA complexes into a specific location in an animal's body. For example, Nabel et al. teach injection via a catheter into the arterial wall. In another example, the inventors have used intraperitoneal injection to allow for gene transfer into mice.

The present invention also contemplates compositions comprising a liposomal complex. This liposomal complex will comprise a lipid component and a DNA segment encoding a nucleic acid encoding a mutant form of Bik. The nucleic acid encoding the mutant form of Bik employed in the liposomal complex can be, for example, one that encodes Bik-T145A or Bik-T145D.

The lipid employed to make the liposomal complex can be any of the above-discussed lipids. In particular, DOTMA, DOPE, and/or DC-Chol may form all or part of the liposomal complex. The inventors have had particular success with complexes comprising DC-Chol. In a preferred embodiment, the lipid will comprise DC-Chol and DOPE. While any ratio of DC-Chol to DOPE is anticipated to have utility, it is anticipated that those comprising a ratio of DC-Chol:DOPE between 1:20 and 20:1 will be particularly advantageous. The inventors have found that liposomes prepared from a ratio of DC-Chol:DOPE of about 1:10 to about 1:5 have been useful.

In a specific embodiment, one employs the smallest region needed to enhance retention of Bik in the nucleus of a cell so that one is not introducing unnecessary DNA into cells which receive a Bik gene construct. Techniques well known to those of skill in the art, such as the use of restriction enzymes, will allow for the generation of small regions of Bik. The ability of these regions to inhibit neu can easily be determined by the assays reported in the Examples.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

D. Electroporation

In certain embodiments of the present invention, the expression construct is introduced into the cell via electroporation. Electroporation involves the exposure of a suspension of cells and/or DNA to a high-voltage electric discharge.

Transfection of eukaryotic cells using electroporation has been quite successful. Mouse pre-B lymphocytes have been transfected with human kappa-immunoglobulin genes (Potter et al., 1984), and/or rat hepatocytes have been transfected with the chloramphenicol acetyltransferase gene (Tur-Kaspa et al., 1986) in this manner.

E. Calcium Phosphate and/or DEAE-Dextran

In other embodiments of the present invention, the expression construct is introduced to the cells using calcium phosphate precipitation. HumanKB cells have been transfected with adenovirus 5 DNA (Graham and Van Der Eb, 1973) using this technique. Also in this manner, mouse L(A9), mouse C127, CHO, CV-1, BHK, NIH3T3 and/or HeLa cells were transfected with a neomycin marker gene (Chen and Okayama, 1987), and/or rat hepatocytes were transfected with a variety of marker genes (Rippe et al., 1990).

In another embodiment, the expression construct is delivered into the cell using DEAE-dextran followed by polyethylene glycol. In this manner, reporter plasmids were introduced into mouse myeloma and/or erythroleukemia cells (Gopal, 1985).

F. Particle Bombardment

Another embodiment of the invention for transferring a naked DNA expression construct into cells may involve particle bombardment. This method depends on the ability to accelerate DNA-coated microprojectiles to a high velocity allowing them to pierce cell membranes and/or enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten and/or gold beads.

G. Direct Microinjection and/or Sonication Loading

Further embodiments of the present invention include the introduction of the expression construct by direct microinjection and/or sonication loading. Direct microinjection has been used to introduce nucleic acid constructs into Xenopus oocytes (Harland and Weintraub, 1985), and/or LTK-fibroblasts have been transfected with the thymidine kinase gene by sonication loading (Fechheimer et al, 1987).

H. Adenoviral Assisted Transfection

In certain embodiments of the present invention, the expression construct is introduced into the cell using adenovirus assisted transfection. Increased transfection efficiencies have been reported in cell systems using adenovirus coupled systems (Kelleher and Vos, 1994; Cotten et al, 1992; Curiel, 1994).

### V. Combination Treatments

In order to increase the effectiveness of a therapeutic gene product encoded by a construct comprising a promoter of the invention, it may be desirable to combine these compositions with other agents effective in the treatment of hyperproliferative disease, such as anti-cancer agents. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This process may involve contacting the cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

Therapy with the methods and compositions of the present invention can be used in conjunction with chemotherapeutic, radiotherapeutic, immunotherapeutic therapy, surgery, hormonal therapy, or additional gene therapy with other pro-apoptotic or cell cycle regulating agents. Gene therapy with the inventive promoters and/or gene therapy in addition to the inventive compositions and methods may utilize inducers of cellular proliferation; antisense sequences for inducers of cellular proliferation; inhibitors of cellular proliferation, such as p53, p16, Rb, APC, DCC, NF-1, NF-2, WT-1, MEN-I, MEN-II, zac1, p73, VHL, MMAC1/PTEN, DBCCR-1, FCC, rsk-3, p27, p27/p16 fusions, Bik/p27 fusions, anti-thrombotic genes (e.g., COX-1, TFPI), PGS, Dp, E2F, ras, myc, neu, raf, erb, fins, trk, ret, gsp, hst, abl, E1A, p300, genes involved in angiogenesis (e.g., VEGF, FGF, thrombospondin, BAI-1, GDAIF, or their receptors) or MCC; and/or regulators of programmed cell death, such as those that counteract Bcl-2 function and promote cell death (e.g., Bax, Bak, Bik, Bim, Bid, Bad, Harakiri).

### VI. Pharmaceutical Preparations

Pharmaceutical compositions of the present invention comprise an effective amount of a construct comprising control sequences of the present invention that regulate expression of a therapeutic gene product and, in specific embodiment one or more additional agents, dissolved or dispersed in a pharmaceutically acceptable carrier or excipient. An effective amount of a construct is an amount that is capable of retarding or halting cancer cell proliferation, in specific embodiments. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one construct comprising the inventive control sequences that regulate expression of a therapeutic polynucleotide and, in some embodiments one or more additional active ingredients, will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated. In a specific embodiment, the mutant Bik composition is administered in a liposome.

The therapeutic construct comprising the tissue-specific control sequences may comprise different types of carriers depending on whether it is to be administered in solid, liquid or aerosol form, and whether it need to be sterile for such routes of administration as injection. The present invention can be administered intravenously, intradermally, intraarterially, intraperitoneally, intralesionally, intracranially, intraarticularly, intraprostaticaly, intrapleurally, intratracheally, intranasally, intravitreally, intravaginally, rectally, topically, intratumorally, intramuscularly, intraperitoneally, subcutaneously, intravesicularlly, mucosally, intrapericardially, orally, topically, locally, using aerosol, injection, infusion, continuous infusion, localized perfusion bathing target cells directly, via a catheter, via a lavage, in cremes, in lipid compositions (e.g. liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 micrograin/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The therapeutic construct may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trim ethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In other embodiments, one may use eye drops, nasal solutions or sprays, aerosols, mouthwashes, or inhalants in the present invention. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in preferred embodiments the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments the construct comprising the therapeutic polynucleotide, such as the Bik mutant form, is prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, mouthwashes, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain preferred embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Additional formulations which are suitable for other modes of administration include suppositories. Suppositories are solid dosage forms of various weights and shapes, usually medicated, for insertion into the rectum, vagina or urethra. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle that contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin or combinations thereof.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Breast Cancer Tissue-Specific Expression

Current breast cancer (BC) therapies, such as chemotherapy (CT) and radiotherapy, have low selectivity for tumor cells and side effects for normal tissues. To minimize the side effects, these therapies are generally given in an intermittent manner, allowing normal cells to recover between treatment cycles. However, during the recovery period, some surviving cancer cells become more resistant to the treatment because of gene mutation. Consequently, cancer recurrence or progression may occur. Tumor-targeting gene therapy can minimize treatment side effects and the risk of developing resistance by acting on the tumor-specific signaling pathways. In the present embodiment, breast cancer-specific promoters are used for breast cancer-targeting gene therapy of an exemplary therapeutic polynucleotide, mutant Bik.

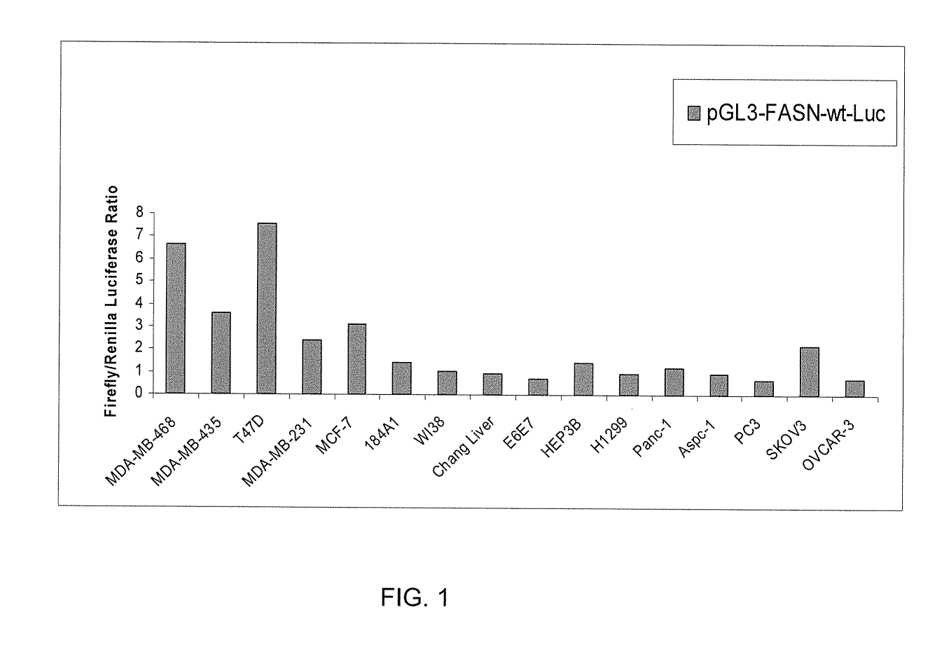

FIG. 1 shows transient luciferase expression of fatty acid synthase (FASN) promoter in human normal and cancer cell lines. Cells (1×10⁶) were transfected with 2 μg pGl3-FASN-luciferase vector, as well as 0.2 μg pRL-TK as internal standards by electroporation. The luciferase activity was measured after 24 hrs. The activity of FASN promoter was highly expressed in exemplary breast cancer cell lines, such as, T47D, MDA-MB-435, and its expression was low in normal and other cancer cell lines. Those results showed FASN promoter had certain breast cancer specificity.

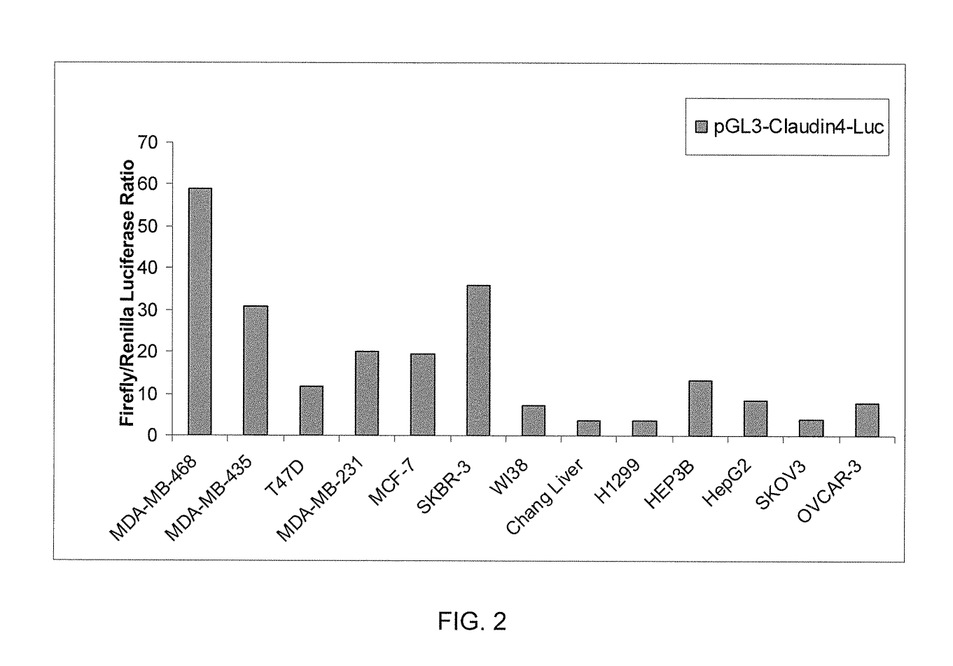

In FIG. 2, the promoter activities of tight junction protein Claudin 4 in human normal and cancer cells is shown. Cells (1×10⁶) were transfected with 2 μg pGl3-claudin 4 luciferase vector, as well as 0.2 μg pRL-TK as internal standards by electroporation. The luciferase activity was measured after 24 hrs. The activity of claudin 4 promoter was highly expressed in breast cancer cell lines, and its expression was relatively low in normal and other cancer cell lines. The results showed Claudin 4 promoter had relative specificity for breast carcinomas.

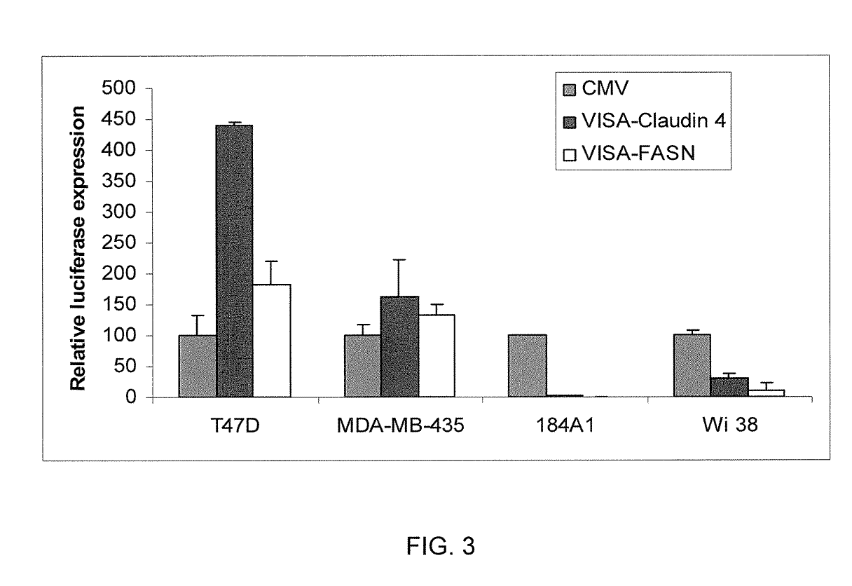

FIG. 3 shows the promoter activities of WPRE+TSTA (VISA)-enhanced claudin 4 and fatty acid synthase in breast cancer cells and normal cells in vitro. Cells (1×10⁶) were transfected with 2 μg VISA-claudin 4 or VISA-fatty acid synthase promoter vector, as well as 0.2 μg pRL-TK as internal standards by electroporation. The luciferase activity was measured after 24 hrs. The activity of VISA-enhanced claudin 4 and fatty acid synthase promoters were highly expressed in T47D and MDA-MB-435 human breast cancer cell lines, higher or comparable with CMV promoter, while its expression still remained low in 184A1 and Wi38 normal cell lines. The results showed claudin 4 and fatty acid synthase promoters were dominantly expressed in breast cancer cells, and kept their specificity after enhanced by VISA system.

### Example 2

### Ovarian Cancer-Specific Promoters

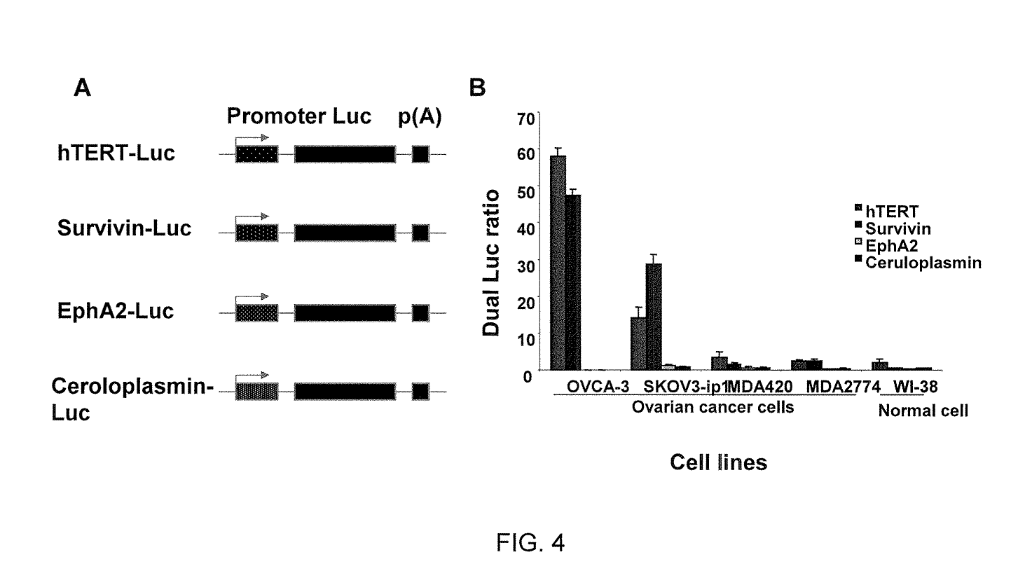

FIGS. 4A-4B show activity of selective promoters in ovarian cancer cell lines and normal cells. In FIG. 4, hTERT and Survivin promoters are active in ovarian cancer. In FIG. 4A, there are exemplary constructs of candidates for ovarian cancer promoter, including a diagram of the promoter-driven luciferase report plasmids. In FIG. 4B, a panel of ovarian cancer cell lines and normal lung fibroblast cells (WI-38) were transiently co-transfected with plasmid DNA indicated and pRL-TK. Forty-eight hours later, dual luciferase ratio was measured and shown as RLU (ratio) normalized to the Renilla luciferase control. The data represent the mean of four independent experiments; bar, SD.

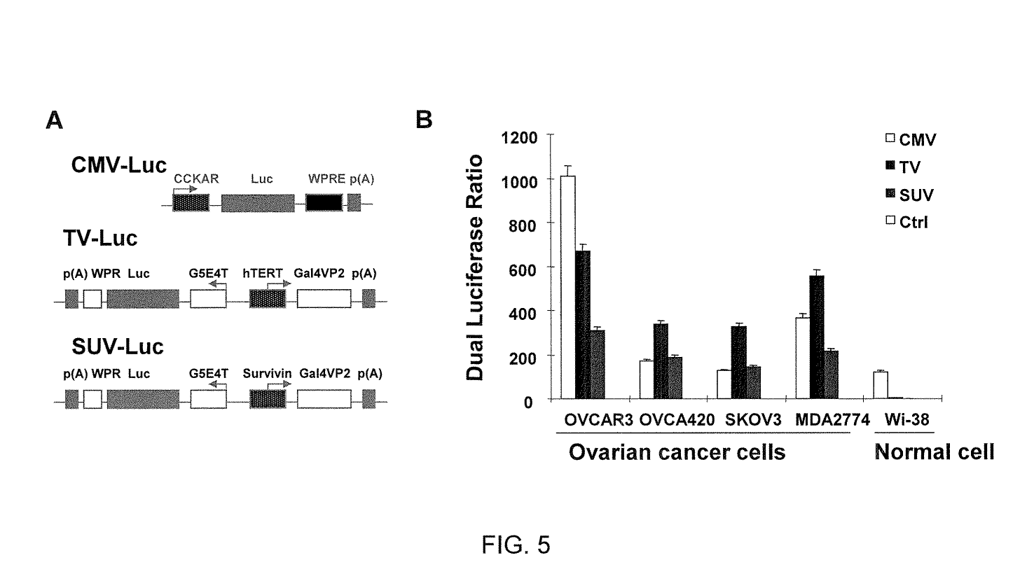

FIGS. 5A-5B show comparison of CMV, TV and SUV promoter activities in ovarian cancer cell lines and normal cells. In FIG. 5A, there is a schematic diagram of exemplary engineered hTERT- and survivin-based constructs in the pGL3 backbone (VISA, VP16-GAL4 intergrated systemic amplifier; TV, hTERT-VISA; SUV; Survivin-VISA). In FIG. 5B, activity of CMV, TV and SUV promoter activities in ovarian cancer cell lines and normal cells is demonstrated. Ovarian cancer cell lines (OVCAR3, OVCA420, SKOV3, MDA2774) and normal cells (WI-38) were transiently cotransfected with the indicated plasmid DNA and pRL-TK. Forty-eight hours later, dual luciferase ratio was measured and shown as RLU (ratio) normalized to the Renilla luciferase control. The data represent the mean of four independent experiments.

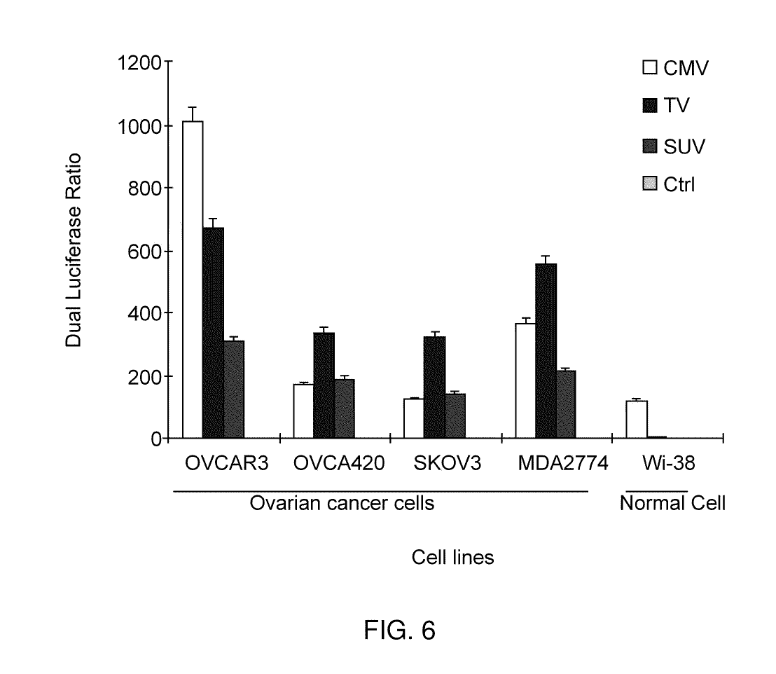

FIG. 6 demonstrates that hTert-VISA (TV) is specifically expressed in ovarian cancer cells but not in normal cells. TV comprises the hTert-VISA promoter (VISA=WPRE+TSTA), SUV is survivin-VISA, and the figure shows that the TV promoter drives expression of luciferase in ovarian cancer cells but not in the exemplary normal fibroblasts.

### Example 3

### Ovarian Cancer-Specific Expression or Breast Cancer-Specific

The present embodiments utilized ovarian cancer-specific promoter sequences to control expression of a therapeutic polynucleotide, for example a mutant Bik polypeptide. Exemplary methods and compositions directed to this goal are described in this Example. Although this example refers to ovarian cancer-specific expression this is merely an illustrative embodiment and one of skill in the art recognizes that this exemplary description may be applied analogously to other embodiments, such as for breast cancer.

An exemplary embodiment of WPRE enhancer may be released from pGEM-3Z-WPRE by Asp718/SalI digestion and inserted into the Small sites of pGL3-basic by blunt ligation to produce intermediate pGL3-Luc-WPRE. Plasmid pGL3-basic may then be digested with XbaI, Klenow blunted and annealed to the blunted Asp718/SalI WPRE fragment of intermediate pGL3-Luc-WPRE to give pGL3-Luc-WPRE. The ovarian tissue-specific regulatory region may be subcloned into blunted NheI/XhoI site of pGL3-Luc-WPRE.

### Transfection

Cells may be seeded in 12-well plates at 40-50% confluence at 37° C. with 5% CO₂ in corresponding medium as described above, 16 h prior to transfection. Cells may be transfected with designated plasmid DNA along with pRL-TK as internal control, using DOTAP:Chol liposome (from N. Templeton, Baylor College of Medicine, Houston, Tex.) according to the recommended method. The non-expression vector, pGL3-basic, was used as a negative control. To compare the activities of transcriptional regulatory elements with each other, the same molar amount of plasmid DNA was used.

### Orthotopic Animal Models of Ovarian Cancer and Systemic Plasmid DNA Delivery

Athymic female BALB/c nu/nu mice (Charles River Laboratories, Wilmington, Mass.), at 6-8 weeks of age, were used as xenograft hosts. Mice were maintained in a specific pathogen-free environment, in compliance with M.D. Anderson Cancer Center rules. Ovarian cancer cells in logarithmic-phase growth may be trypsinized and washed twice with PBS. For the orthotopic model, mice may be anesthetized with Aventin (Sigma) (Xie, Mol. Endocrinl 2004) and placed at the supine position. The abdomen area may be cleaned with 70% ethanol, and an upper midline abdomen incision may be made. An ovary may be exteriorized and a tail may be injected with 50 μl of aliquots of the appropriate ovarian cancer cells (1×10⁶ cells, for example). The incision may be closed with wound clips.

Plasmid DNA:liposome complexes may be prepared as previously described (Templeton, Nat Biotech 1997). Briefly, DNA and DOTOP:Chol stock may be separately diluted in 5% dextrose in water (D5W) at room temperature. The DNA solution may be added rapidly at the surface of the liposome solution in equal volume and mixed by pipetting up and down twice. The preparation may be made fresh 2 h prior to injection. The nude mice in which tumors reached about 50 mm³ in ectopic model or the same period of time in orthotopic model may be injected with 100 μl of DNA:liposome complexes containing 50 μg of DNA into the tail vein using a 29-gauge needle, once a day for three consecutive days. Mice may be in vivo imaged every day post injection and sacrificed 24 h after last injection.

### Luciferase Assays

Transiently transfected cells may be lysed and assayed for luciferase activity by using the Dual-Luciferase® Reporter Assay System (Promega, Madison, Wis.) following the manufacturer protocol with a TD 20/20 luminometer (Turner Designs, Sunnyvale, Calif.). The dual luciferase ratio may be defined as the Firefly luciferase activity of the tested plasmids over the Renilla luciferase activity of pRL-TK, expressed as the means of triplicate transfections, which may be repeated at least four times. Compared to the ratio of CMV activity, the percentage may be presented.

To assay tissue-derived luciferase activity, animals may be euthanized and dissected. Tissue specimens from tumors and other organs including pancreas, lung, heart, liver, spleen, kidney, brain, intestine, muscle, and ovary, et al. may be resected, and homogenized with a PRO250 homogenizer (Pro Scientific, Inc., Monore, Conn.) in 300 μl of luciferase lysis buffer (Promega) containing 1/100 diluted protein inhibitor cocktail (Roche). Specimens were centrifuged at 8,000 rpm for 5 min and placed temporarily on ice. Luciferase activity of the supernatants may be measured with a Lumat LB9507 luminometer (Berthod, Bad Wildbad, Germany) and the protein concentration may be determined using the detergent compatible (DC) protein assay system (Bio-Rad, Hercules, Calif.) with MRX microplate reader (Dynex technologies, Inc., Chantilly, Va.). The luminescence results may be reported as relative light units (RLU) per milligram of protein.

### Imaging and Quantification of Bioluminescence Data

Mice may be anaesthetized with Aventin. D-luciferin (Xenogen, Alemeda, Calif.) (30 mg/ml in PBS) that was intraperitoneally injected at 150 mg/kg mouse body weight. Ten min after D-luciferin injection, mice may be imaged with an IVIS™ Imaging System (Xenogen), consisting of a cooled CCD camera mounted on a light-tight specimen chamber (dark box), a camera controller, a camera cooling system, and a Windows-based computer system. Imaging parameters may be maintained for comparative analysis. Gray scale reflected images and bioluminescence colorized imaged may be superimposed and analyzed using the Living Imaging software version 2.11 (Xenogen). A region of interest (ROI) may be manually selected over relevant regions of signal intensity. The area of the ROI may be kept constant and the intensity may be recorded as maximum photon counts within a ROI (Xie et al., 2004). In some experiments after imaging, animals may be euthanized and organs of interest may be removed, arranged on black, bioluminescence-free paper, and ex vivo imaged within 30 min.

In particular embodiments of the present invention, constructs are similarly generated comprising these exemplary ovarian-specific promoters operatively linked to a polynucleotide encoding a mutant Bik, for example, followed by introduction into a mammal in need of ovarian cancer therapy treatment based on analogous methods described herein. Parameters are easily optimized by those of skill in the art, such as delivery mode, concentration of composition, and so forth.

In further embodiments of the present invention, the ovarian cancer-specific elements are narrowed further to identify even smaller segments within that retain ovarian cancer-specific expression activity. For example, deletion constructs may be made of these respective regions, and their tissue specificity is tested to identify the smaller segments that maintain the ability to direct expression in ovarian cancer tissue.

### Two-Step Transcription Amplification (TSTA) Significantly Increases Gene Expression

The hTERT promoter increases the safety and effectiveness of gene therapy. However, the activity of this unmodified hTERT promoter is much weaker than that of commonly used non-tissue-specific virus-based promoters, such as the cytomegalovirus (CMV) promoter (Cong, Wen et al. 1999; Gu, Andreeff et al. 2002; Komata, Kondo et al. 2002). One of the amplification approaches using the GAL4-VP16 fusion protein, called a two-step transcriptional amplification (activation) (TSTA) approach, can potentially be used to augment the transcriptional activity of cellular promoters (Iyer, Wu et al. 2001; Zhang, Adams et al. 2002). In this system, the first step involves the tissue-specific expression of the GAL4-VP16 fusion protein. In the second step, GAL4-VP16, in turn, drives target gene expression under the control of GAL4 response elements in a minimal promoter. The use of TSTA can potentially lead to amplified levels of the transgene expression.

### WPRE is a Useful Enhancer

To increase the activity of tissue-specific promoters, the present inventors and others may use CMV enhancer fused to a minimal tissue-specific promoter. Though the activity may be increased, the tissue specificity may be decreased. To address this issue, in specific embodiments the post-transcriptional regulatory element of the woodchuck hepatitis virus (WPRE) may be employed, which involves modification of RNA polyadenylation, RNA export, and/or RNA translation (Donello, Loeb et al., 1998). Enhancement of WPRE occurred both during transient expression in non-viral vectors and viral vectors (Loeb, Cordier et al., 1999; Glover, Bienemann et al. 2002) and when the gene is stably incorporated into the genome of target cells with no loss of tissue specificity (Lipshutz, Titre et al. 2003). WPRE in the sense orientation cloned between the target gene and the poly(A) sequence stimulated 2- to 7-fold more luciferase expression in vitro and 2- to 50-fold in vivo without the use of the WPRE (Zufferey, Donello et al., 1999; Lipshutz, Titre et al., 2003). Furthermore, long-term transgene expression can be mediated by WPRE-containing adenoviral vectors (Glover, Bienemann et al., 2003). Therefore, the WPRE is an effective tool for increasing and prolonging the expression of transgenes in gene therapy.

TSTA and WPRE Enhance the Activity of hTERT Promoter

To determine whether TSTA and WPRE enhance the activity of hTERT promoter, the present inventors first subcloned a series of TSTA- and WPRE-containing hTERTp-based promoter composites. The hTERTp fragment (nt −378 to +56) (Takakura, Kyo et al., 1999) was PCR-amplified from the DNA extracts of LNCaP cells. The hTERTp fragment was subcloned into pGL3-Basic plasmid to drive the firefly luciferase gene, leading to phTERTp-Luc. The WPRE was then inserted into phTERTp-Luc-Luc, resulting in phTERTp-Luc-WPRE. To employ the TSTA system, hTERTp was substituted for PSA promoter of pTSTA plasmid (Zhang, Johnson et al., 2003), producing phTERTp-TSTA-Luc. Finally, the plasmid phTERTp-TSTA-Luc-WPRE was obtained by inserting the WPRE fragment into phTERTp-TSTA-Luc.

In specific embodiments of the invention, TSTA and WPRE enhance the activity of the hTERT promoter.

### Example 4

### In Vitro Testing of Cancer-Specific Promoters

A construct(s) comprising the inventive promoters operably linked to a respective therapeutic polynucleotide are tested in vitro. For example, the control sequences are selected, in some embodiments based on previously generated data suggesting the sequence is effective in a desired tissue or cell. In other embodiments, control sequences are selected without prior knowledge of potential effectiveness. The control sequence to be tested is operably linked to a reporter sequence, such as one whose expression and/or gene product may be monitored, including by color, light, or fluorescence, for example. Examples of reporter genes include luciferase or β-galactosidase. Additional control sequences of any kind may also be added to the construct, including transcriptional or post-transcriptional control sequences, minimal promoters, and so forth. The construct to be tested and its one or more appropriate controls are then introduced into a desired cell and assayed for expression. In particular embodiments, the construct to be tested generates expression at such levels as determined by the skilled artisan to be effective in the desired cell or tissue in which it resides.

### Example 5

### In Vivo Testing of Cancer-Specific Promoters

A construct(s) comprising the inventive promoters operably linked to a respective therapeutic polynucleotide as it relates to its anti-tumor activity is tested in an animal study. The construct is delivered by a vector, such as in a liposome or on a plasmid or viral vector, into nude mice models to test for its anti-tumor activity. Once the anti-tumor activity is demonstrated, potential toxicity is further examined using immunocompetent mice, followed by clinical trials.

In a specific embodiment, the preferential growth inhibitory activity of a construct comprising the inventive promoter operably linked to a therapeutic polynucleotide is tested in an animal. Briefly, and by example only, HER-2/neu overexpressing breast cancer cell lines (such as SKBR3 and MDA-MB361) are administered into mammary fat-pad of nude mice to generate a breast xenografted model. After the tumors reach a particular size, the construct of the present invention or its control is intravenously injected into the mouse in an admixture with an acceptable carrier, such as liposomes. The tumor sizes and survival curve from these treatments are compared and statistically analyzed. In a preferred embodiment, the constructs comprising the promoters of the invention preferentially inhibit the growth of a tumor tissue.

### Example 6

### Clinical Testing with Cancer-Specific Promoters

This example is concerned with the development of human treatment protocols using constructs comprising the cancer-specific promoters of the invention alone or in combination with other anti-cancer drugs. The anti-cancer drug treatment using constructs comprising the cancer-specific promoters of the invention will be of use in the clinical treatment of various cancers. Such treatment will be particularly useful tools in anti-tumor therapy, for example, in treating patients with the respective breast and ovarian cancers, such as those that are resistant to conventional chemotherapeutic regimens.

The various elements of conducting a clinical trial, including patient treatment and monitoring, will be known to those of skill in the art in light of the present disclosure. The following information is being presented as a general guideline for use in establishing the constructs comprising the cancer-specific promoters of the invention in clinical trials.

Patients with advanced, metastatic breast or ovarian cancers chosen for clinical study will typically be at high risk for developing the cancer, will have been treated previously for the cancer which is presently in remission, or will have failed to respond to at least one course of conventional therapy. In an exemplary clinical protocol, patients may undergo placement of a Tenckhoff catheter, or other suitable device, in the pleural or peritoneal cavity and undergo serial sampling of pleural/peritoneal effusion. Typically, one will wish to determine the absence of known loculation of the pleural or peritoneal cavity, creatinine levels that are below 2 mg/dl, and bilirubin levels that are below 2 mg/dl. The patient should exhibit a normal coagulation profile.

In regard to the constructs comprising the cancer-specific promoters of the invention and other anti-cancer drug administration, a Tenckhoff catheter, or alternative device may be placed in the pleural cavity or in the peritoneal cavity, unless such a device is already in place from prior surgery. A sample of pleural or peritoneal fluid can be obtained, so that baseline cellularity, cytology, LDH, and appropriate markers in the fluid (CEA, CA15-3, CA 125, PSA, p38 (phosphorylated and un-phosphorylated forms), Akt (phosphorylated and un-phosphorylated forms) and in the cells (constructs comprising the cancer-specific promoters of the invention) may be assessed and recorded.

In the same procedure, the constructs comprising the cancer-specific promoters of the invention may be administered alone or in combination with the other anti-cancer drug. The administration may be in the pleural/peritoneal cavity, directly into the tumor, or in a systemic manner, for example. The starting dose may be about 0.05 mg/kg body weight. Three patients may be treated at each dose level in the absence of grade>3 toxicity. Dose escalation may be done by 100% increments (0.5 mg, 1 mg, 2 mg, 4 mg) until drug related grade 2 toxicity is detected. Thereafter dose escalation may proceed by 25% increments. The administered dose may be fractionated equally into two infusions, separated by six hours if the combined endotoxin levels determined for the lot of the constructs comprising the cancer-specific promoters of the invention, and the lot of anti-cancer drug exceed 5 EU/kg for any given patient.

The constructs comprising the cancer-specific promoters of the invention and/or the other anti-cancer drug combination, may be administered over a short infusion time or at a steady rate of infusion over a 7 to 21 day period. The constructs comprising the cancer-specific promoters of the invention infusion may be administered alone or in combination with the anti-cancer drug. The infusion given at any dose level will be dependent upon the toxicity achieved after each. Hence, if Grade II toxicity was reached after any single infusion, or at a particular period of time for a steady rate infusion, further doses should be withheld or the steady rate infusion stopped unless toxicity improved. Increasing doses of the constructs comprising the cancer-specific promoters of the invention, in combination with an anti-cancer drug will be administered to groups of patients until approximately 60% of patients show unacceptable Grade III or IV toxicity in any category. Doses that are ⅔ of this value could be defined as the safe dose.

Physical examination, tumor measurements, and laboratory tests should, of course, be performed before treatment and at intervals of about 3-4 weeks later. Laboratory studies should include CBC, differential and platelet count, urinalysis, SMA-12-100 (liver and renal function tests), coagulation profile, and any other appropriate chemistry studies to determine the extent of disease, or determine the cause of existing symptoms. Also appropriate biological markers in serum should be monitored e.g. CEA, CA 15-3, p38 (phosphorylated and non-phosphorylated forms) and Akt (phosphorylated and non-phosphorylated forms), p185, etc.

To monitor disease course and evaluate the anti-tumor responses, it is contemplated that the patients should be examined for appropriate tumor markers every 4 weeks, if initially abnormal, with twice weekly CBC, differential and platelet count for the 4 weeks; then, if no myelosuppression has been observed, weekly. If any patient has prolonged myelosuppression, a bone marrow examination is advised to rule out the possibility of tumor invasion of the marrow as the cause of pancytopenia. Coagulation profile shall be obtained every 4 weeks. An SMA-12-100 shall be performed weekly. Pleural/peritoneal effusion may be sampled 72 hours after the first dose, weekly thereafter for the first two courses, then every 4 weeks until progression or off study. Cellularity, cytology, LDH, and appropriate markers in the fluid (CEA, CA15-3, CA 125, ki67 and Tunel assay to measure apoptosis, Akt) and in the cells (Akt) may be assessed. When measurable disease is present, tumor measurements are to be recorded every 4 weeks. Appropriate radiological studies should be repeated every 8 weeks to evaluate tumor response. Spirometry and DLCO may be repeated 4 and 8 weeks after initiation of therapy and at the time study participation ends. An urinalysis may be performed every 4 weeks.

Clinical responses may be defined by acceptable measure. For example, a complete response may be defined by the disappearance of all measurable disease for at least a month. Whereas a partial response may be defined by a 50% or greater reduction of the sum of the products of perpendicular diameters of all evaluable tumor nodules or at least 1 month with no tumor sites showing enlargement. Similarly, a mixed response may be defined by a reduction of the product of perpendicular diameters of all measurable lesions by 50% or greater with progression in one or more sites.

### Example 7

### Fatty Acid Synthase Promoter was More Selectively Expressed in Breast Cancer Cell Lines than Normal and Other Cancer Cell Lines by Transient Luciferase Assay

Fatty acid synthase promoter (FASN) expression is characterized in breast cancer cell lines compared to normal and other cancer cell lines by transient luciferase assay.

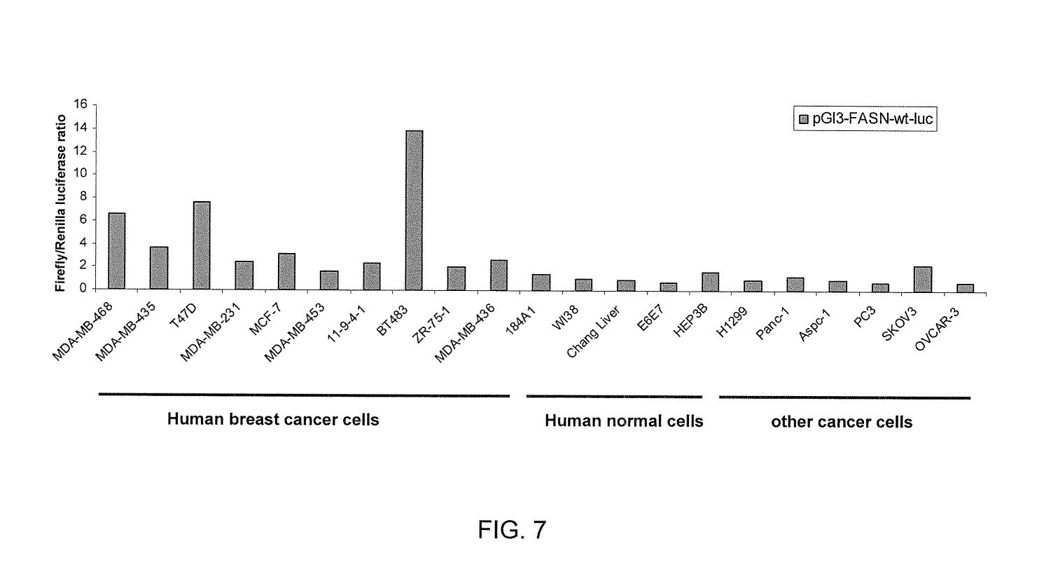

As shown in FIG. 7, the activities of Fatty acid synthase promoter were characterized in human normal and cancer cells. 1×10⁶ cells were transfected with 2 μg pGl3-FASN-luciferase vector, as well as 0.2 μg pRL-TK as internal standards by electroporation. The luciferase activity was measured after 24 hrs. The activity of FASN promoter was highly expressed in breast cancer cell lines, such as, T47D and MDA-MB-435, and its expression was low in normal and other cancer cell lines. Those results showed that FASN promoter was selectively expressed in breast cancer cell lines. Therefore, in specific embodiments of the invention, FASN is employed in regulation of expression.

### Example 8

### Claudin 4 Promoter was More Selectively Expressed in Breast Cancer Cell Lines than Normal and Other Cancer Cell Lines by Transient Luciferase Assay

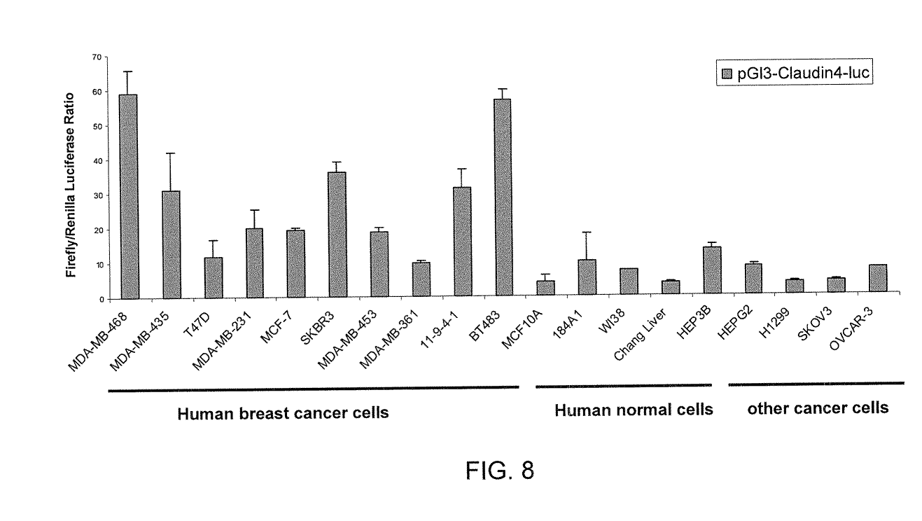

Expression of claudin 4 promoter was ascertained in breast cancer cell lines, normal cell lines, and other cancer cell lines by transient luciferase assay. As shown in FIG. 8, the promoter activities of tight junction protein Claudin 4 in human normal and cancer cells are examined. 1×10⁶ cells were transfected with 2 μg pGl3-claudin 4 luciferase vector, as well as 0.2 μg pRL-TK as internal standards by electroporation. The luciferase activity was measured after 24 hrs. The activity of claudin 4 promoter was highly expressed in breast cancer cell lines, and its expression was relatively low in normal and other cancer cell lines. The results showed Claudin 4 promoter had relative specificity for breast cancer cell lines. Therefore, in particular aspects of the invention, claudin 4 promoter is employed for regulation of expression.

### Example 9

### The Activities of Claudin 4 and Fatty Acid Synthase Promoters Compared to CMV Promoter

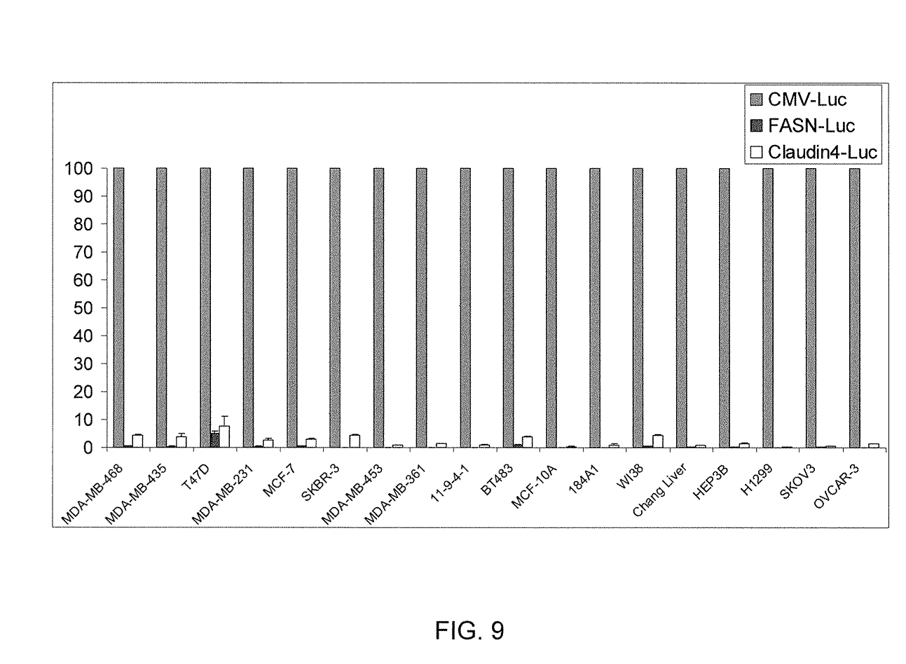

The activities of claudin 4 and fatty acid synthase promoters were compared to CMV promoter. As shown in FIG. 9, the promoter activities of Claudin 4 and fatty acid synthase in human normal and cancer cells are demonstrated. 1×10⁶ cells were transfected with 2 μg pGl3-claudin 4 luciferase vector, as well as 0.2 μg pRL-TK as internal standards by electroporation. The luciferase activity was measured after 24 hrs. The activities of claudin 4 and fatty acid synthase promoters were much weaker than that of CMV promoter, and were about 0.5% and 5% of CMV expression activity, respectively.

### Example 10

### VISA-Enhanced Claudin 4 and Fatty Acid Synthase Promoter

The VISA-enhanced Claudin 4 and fatty acid synthase promoter were characterized in breast cancer cell lines compared to controls. They were strongly expressed in breast cancer cell lines, while remained lowly expressed in normal and other cancer cell lines after 24 hours transient transfection in vitro.

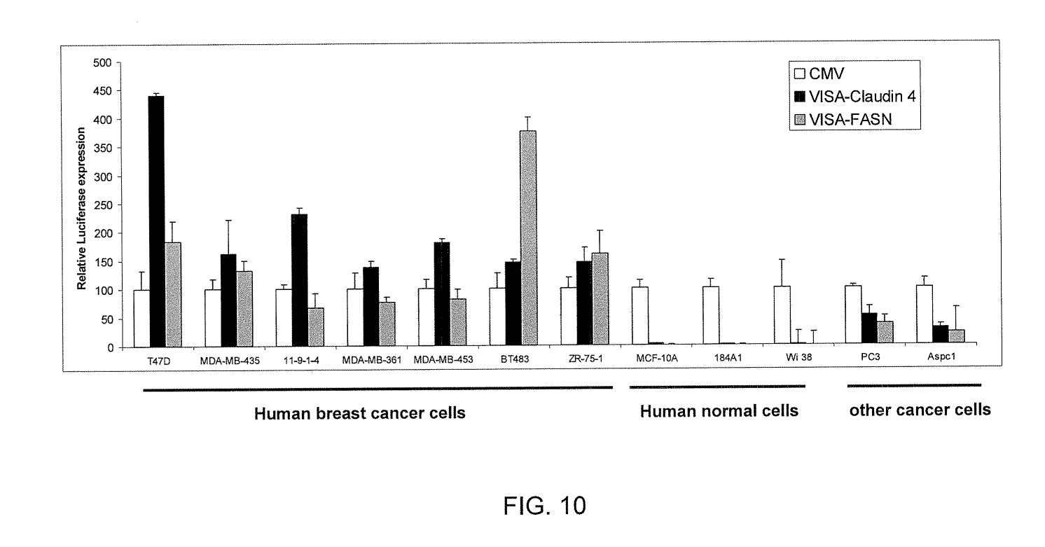

As shown in FIG. 10, the activities of VISA-enhanced claudin 4 and fatty acid synthase promoters in breast cancer and other cell lines. 1×10⁶ cells were transfected with 2 μg pGL3-VISA-Claudin4-Luc or pGL3-VISA-FASN-Luc plasmid, as well as 0.2 μg pRL-TK as internal standards by electroporation. The luciferase activities were measured 24 hrs after transient transfection. The activities of VISA-enhanced claudin 4 and fatty acid synthase promoters were highly expressed in many human breast cancer cell lines, higher or comparable with CMV promoter, while its expression still remained very low in human normal or other cancer cell lines. These results showed that claudin 4 and fatty acid synthase promoters were highly selectively expressed in human breast cancer lines, even after enhanced by VISA system. Thus, such promoters may be employed in certain embodiments of the invention.

### Example 11

### VISA-Claudin4-BIKDD Selectively Inhibits Breast Cancer Cell Lines In Vitro

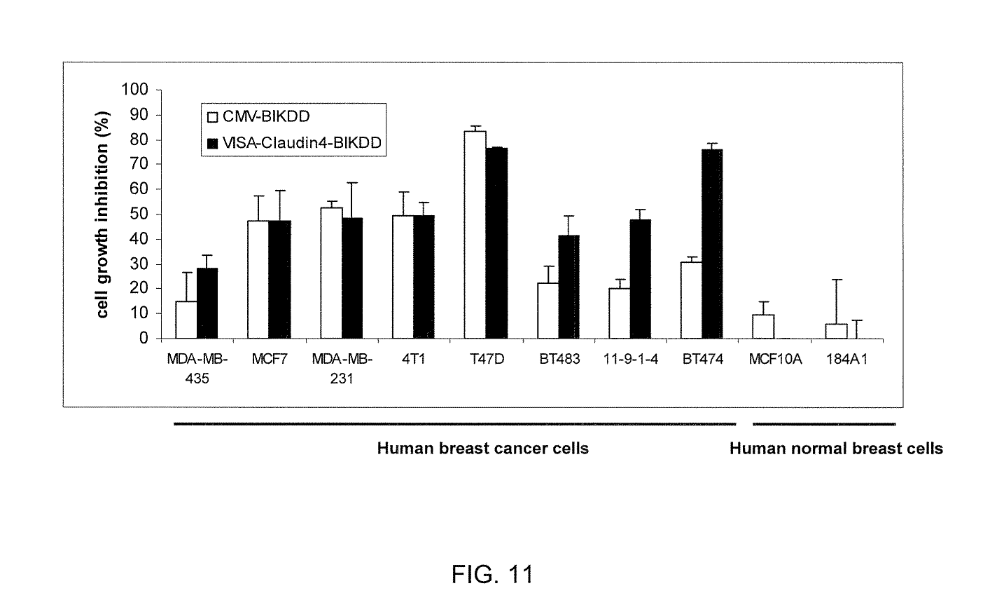

VISA-Claudin4-BIKDD (VISA-claudin4-Bik T33DS35D mutant) was characterized for selective inhibition of breast cancer cell lines in vitro. As shown in FIG. 11, 0.5-1×10⁴ cells were transfected with indicated concentration plasmid by electroporation assay. The cells were incubated with thiazolyl blue tetrazolium bromide for 4 his after 72 hrs, and dissolved with DMSO for 10 min, and measured at OD₅₇₀nm. The cytoxicities of pUK21-VISA-Claudin4-BIKDD were potent in breast cancer cell lines, as comparable as pUK21-CMV-BIKDD, while it showed weak cytoxicity in MCF10A normal breast cell lines.

### Example 12

### The Transient Luciferase Expression of VISA-Claudin4Luc in Breast Cancer Cell Lines by SN Liposome Transfection

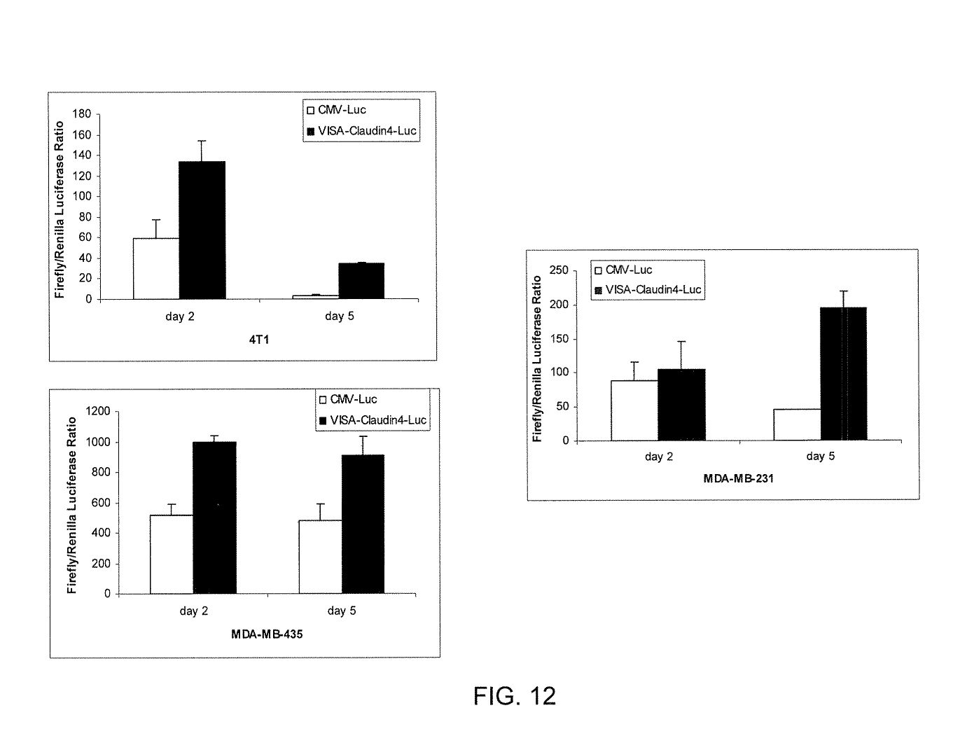

The transient luciferase expression of VISA-Claudin4-Luc in breast cancer cell lines by SN liposome transfection was characterized. As shown in FIG. 12, the activities of VISA-enhanced claudin 4 and fatty acid synthase promoters were characterized in human breast cancer cell lines. 1×10⁶ cells were transfected with 2 μg VISA-claudin 4 promoter vector, as well as 0.2 μg pRL-TK as internal standards by SN liposome transfection. The luciferase activity was measured at indicated times. The activities of VISA-enhanced claudin 4 and fatty acid synthase promoters were highly expressed in MDA-MB-231, MDA-MB-435, and 4T1breast cancer cell lines, and were higher or comparable with CMV promoter in time-dependent manner, as well as other breast cancer cell lines. The results showed that VISA-enhanced claudin 4 and fatty acid synthase promoters were expressed much longer and strongly in breast cancer cells.

### Example 13

### VISA-Enhanced Claudin 4 Promoter in Exemplary 4T1 Breast Cancer in Mice

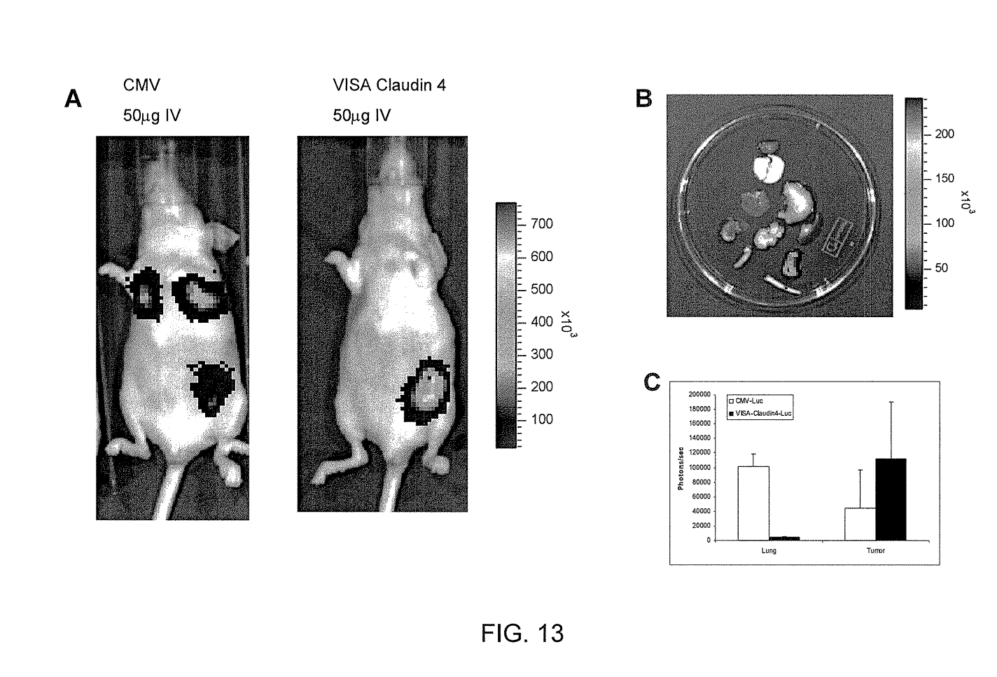

The VISA-enhanced Claudin 4 promoter was strongly expressed in 4T1 breast cancer in mice, while weakly expressed in lung after 48 hours administrated by tail vein. As shown in FIG. 13A, the activities of VISA-enhanced claudin 4 was selectively expressed in 4T1 breast cancer, while CMV promoter was strongly expressed in lung in vivo. In FIG. 13B, the VISA-Claudin4-Luc was strongly expressed in breast carcinoma, while expressed very weakly in other organs of mice. In FIG. 13C, the luciferase expression of VISA-Claudin4-Luc and CMV-luc in lung and tumor were measured by IVIS 100 imaging system, and the data were averaged by 5 mice in each group. 50 μg plasmid plus HLDC liposome were administered into mice by tail vein for one time, and mice were underwent imaging for 1 min with the noninvasive imaging system (IVIS imaging system, xenogen, Alameda, Calif.) after 48 hrs treated with D-luciferins. The promoters of VISA-enhanced claudin 4 were selectively expressed in 4T1 breast cancer, while the CMV promoter was highly expressed in lung. The VISA-enhanced Claudin 4 promoter was strongly expressed in 4T1 breast cancer in mice, while weakly expressed in lung after 48 hours administrated by tail vein.

### Example 14

### Survival Curve of pUK21-VISA-Claudin4-BIKDD and pUK21-VISA-FASN-BIKDD in BALB/CA Mice

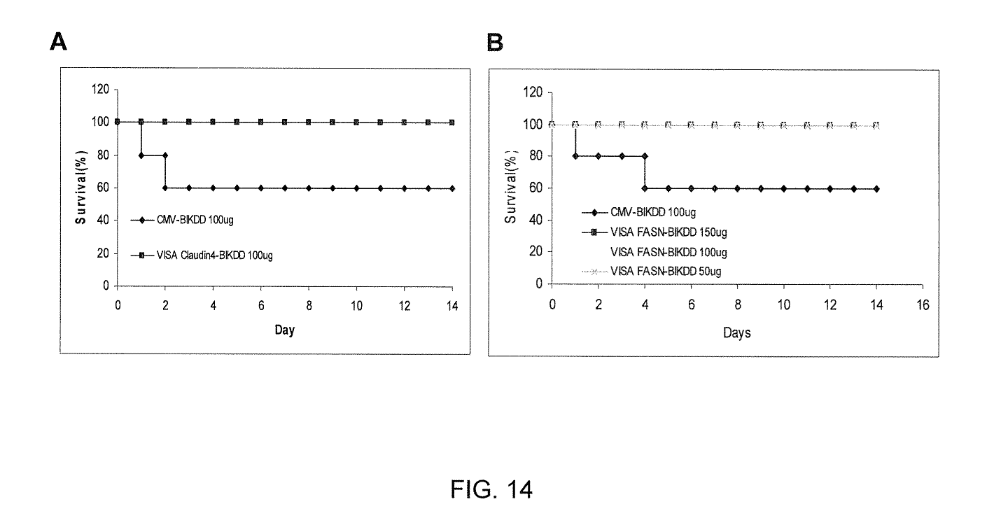

The survival curve of pUK21-VISA-Claudin4-BIKDD and pUK21-VISA-FASN-BIKDD in BALB/cA mice was investigated. In FIG. 14, the acute toxicity of pUK21-VISA-Claudin4-BIKDD and pUK21-VISA-FASN-BIKDD in normal BALB/cA mice is demonstrated. Each mice was injected with indicated concentration plasmid plus HLDC liposome by tail vein, and mice survival were recorded in 14 days. The pUK21-VISA-Claudin4-BIKDD (FIG. 14A) and pUK21-VISA-FASN-BIKDD (FIG. 14B) were tolerenced under dosage of 100 μg/mice without body weight loss.

### Example 15

### VISA-Claudin4-BIKDD Significantly Suppressed the Tumor Growth of Exemplary MDA-Mb-435-Luc Orthotopic Xenografts and 4T1 Orthotopic Synergic Model In Vivo

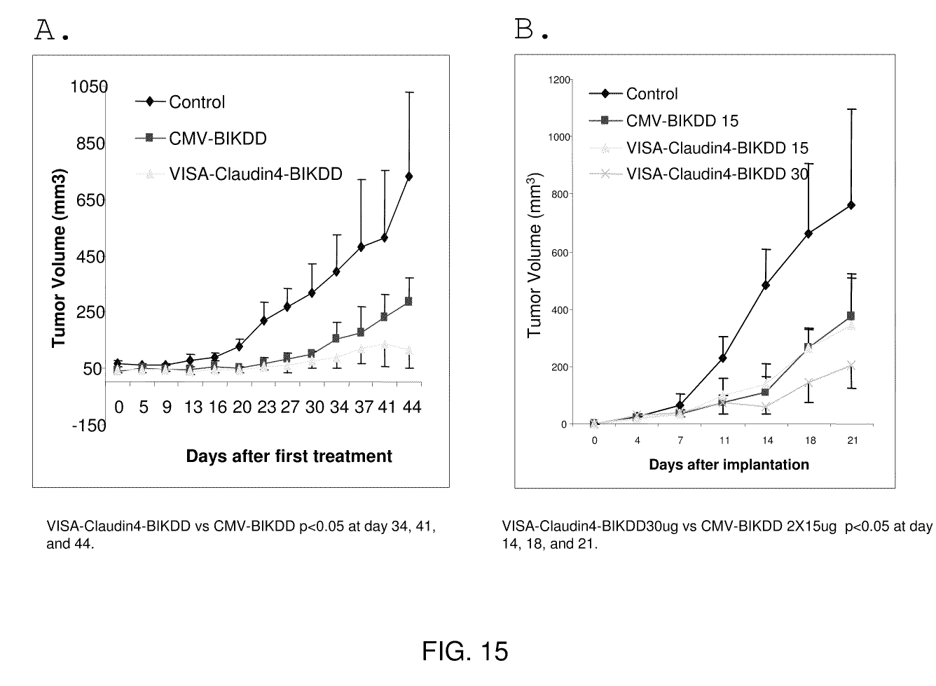

VISA-Claudin4-BIKDD was characterized for impact on tumor growth of exemplary MDA-MB-435-Luc orthotopic xenografts and 4T1 orthotopic synergic model in vivo. As shown in FIG. 15A, the tumor growth of MDA-MB-435 orthotopic xenografts was significantly suppressed by VISA-Claudin4-BIKDD treatment. In FIG. 15B, the tumor growth of 4T1 orthotopic synergic tumor model was significantly suppressed by VISA-Claudin4-BIKDD treatment. 2×10⁶ cells were incubated to the mammary fat pad of female athymic mice or BALB/Ac mice, and the mice were treated with indicated concentration of HLDC and plasmid mixture when the tumor volume reached to 50 mm³. Plasmid plus HLDC liposome were administered into mice by tail vein, and mice were measured tumor volume twice per week, and calculated as following: tumor volume=0.5×length×width×width. VISA-Claudin4-BIKDD can significantly decrease the tumor growth of MDA-MB-435 orthotopic xenografts or 4T1 orthotopic synergic tumor model, which was comparable or better than CMV-BIKDD in vivo.

### Example 16

### VISA-Claudin4-BIKDD in Exemplary MDA-MB-435Luc Orthotopic Xenografts In Vivo

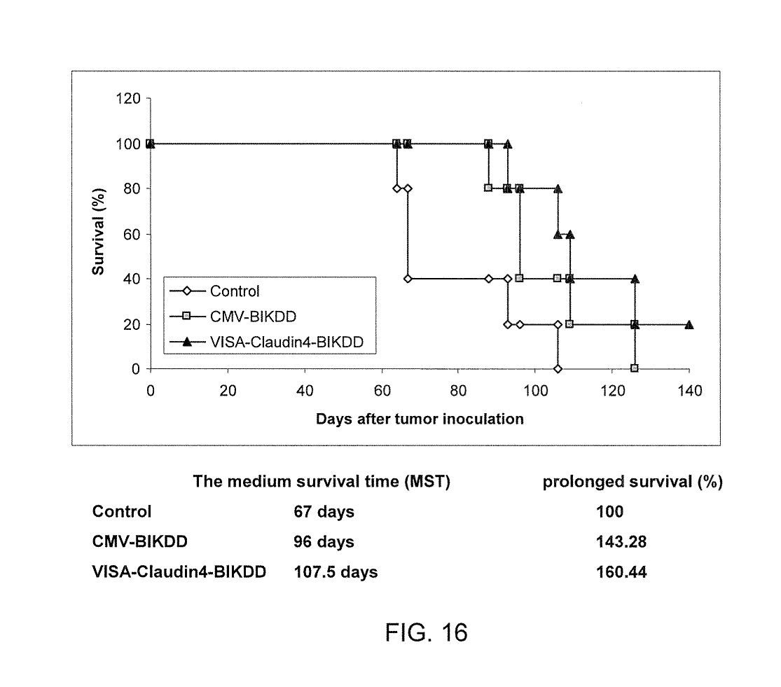

As shown in FIG. 16, VISA-Claudin4-BIKDD greatly prolonged the survival time of MDA-MB-435-Luc orthotopic xenografts in vivo.

### Example 17

### VISA-Claudin4-BIKDD in Combination Therapy

Any promoter embodiments of the present invention may be employed in combination with other anti-cancer therapies, including chemotherapy, radiation, and/or surgery, for example. In specific embodiments, VISA-Claudin4-BikDD is employed in combination with chemotherapeutics such as, for example, lapatinib, Iressa, Tarceva, SAHA, Taxol, Doxorubicin, and/or gemcitabine.

The combination treatment of VISA-Claudin4-BIKDD and chemodrugs in breast cancer cell lines is provided. Ten ng VISA-Claudin4-BIKDD was transfected into 1×10⁶ cells by electroporation, and 1×10⁴ cell were divided into each 96 well with indicated drug concentration. The cells were treated with thiazolyl blue tetrazolium bromide for 4 hr, and measure OD₅₇₀nm after incubation for 72 hrs.

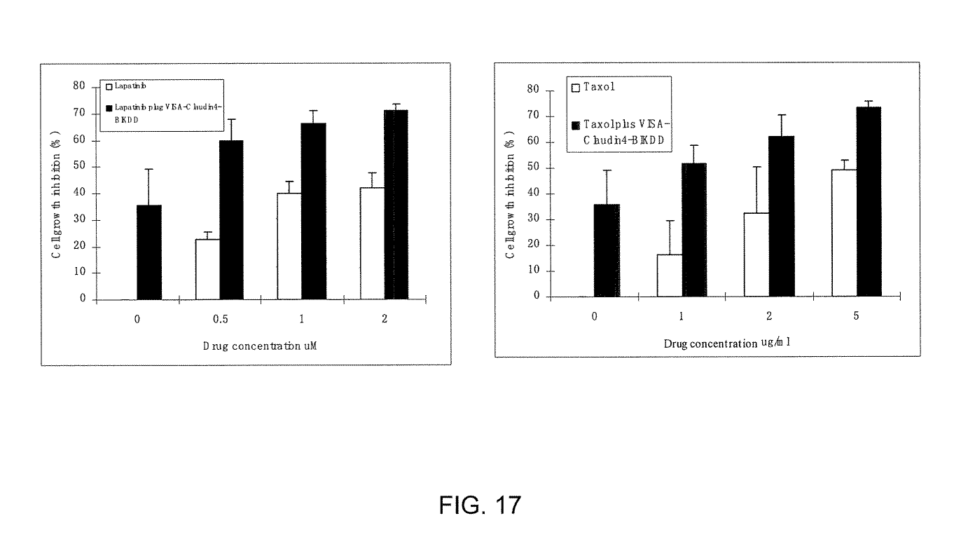

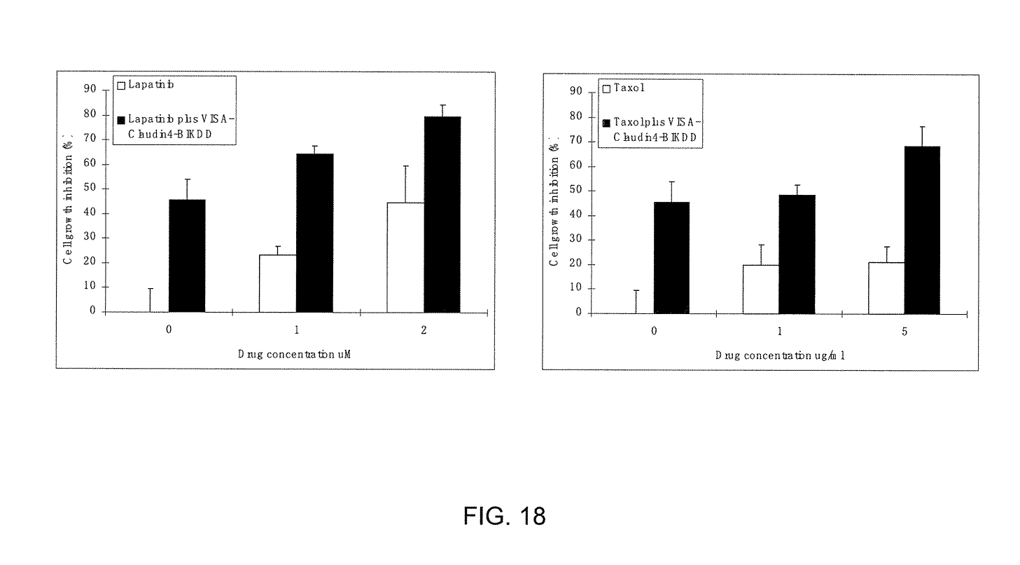

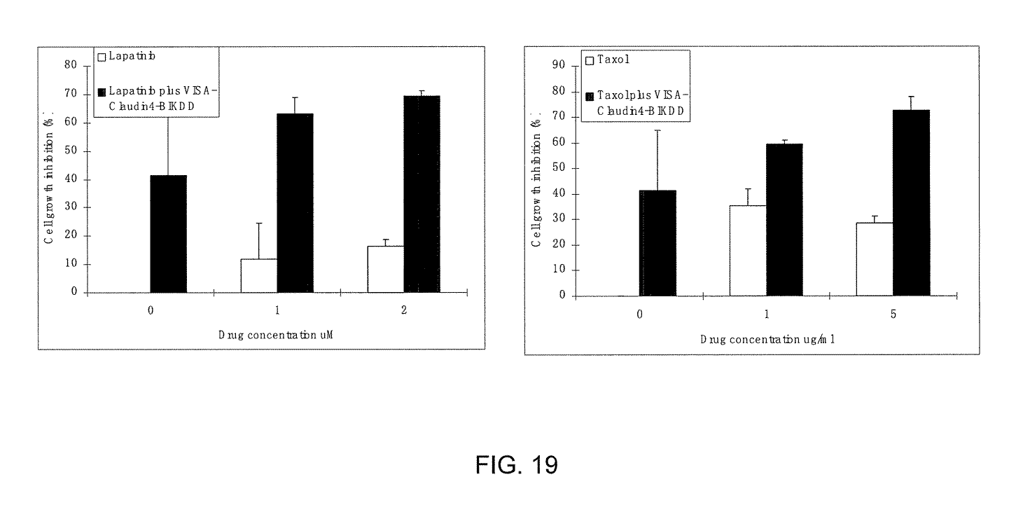

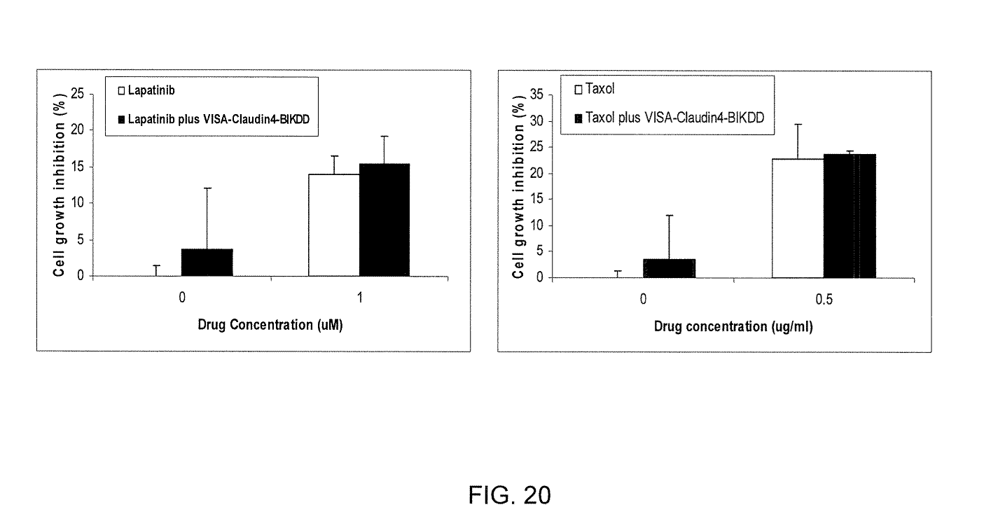

As shown in FIG. 17, VISA-Claudin4-BIKDD has additive combination efficacy with lapatinib and taxol in MDA-MB-453 breast cancer cell line. As shown in FIG. 18, VISA-Claudin4-BIKDD has additive combination efficacy with lapatinib and taxol in MDA-MB-468 breast cancer cell line. As shown in FIG. 19, VISA-Claudin4-BIKDD has additive combination efficacy with lapatinib and taxol in BT474 breast cancer cell line. As shown in FIG. 20, VISA-Claudin4-BIKDD does not promote the cytotoxicity of lapatinib and taxol in MCF10A human breast normal cell line. Thus, it was shown that VISA-Claudin4-BIKDD has additive effects with lapatinib and taxol in human breast cancer cell lines. In other embodiments, there are synergistic effects with compositions of the present invention and other anti-cancer therapies.

### Example 18

### hTERT and Survivin Promoters in Ovarian Cancer

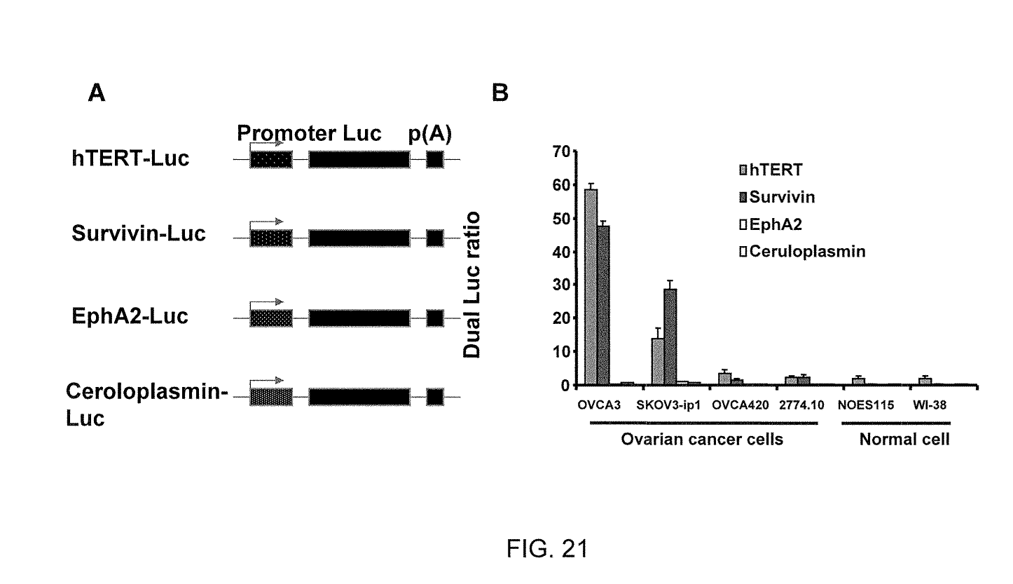

FIG. 21 demonstrates that hTERT and Survivin promoters are active in ovarian cancer. FIG. 21A provides a diagram of the promoter-driven luciferase report plasmids. In FIG. 21B, there is a panel of ovarian cancer cell lines, normal ovarian epithelia cells (NOE115) and fibroblasts (WI-38) that were transiently cotransfected with either the plasmid DNA indicated and pRL-TK. 48 h later, dual luciferase ratio was measured and shown as RLU (ratio) normalized to the Renilla luciferase control. The data represent the mean of four independent experiments. Bar, SD.

### Example 19

### T-VISA is Robust in Ovarian Cancer

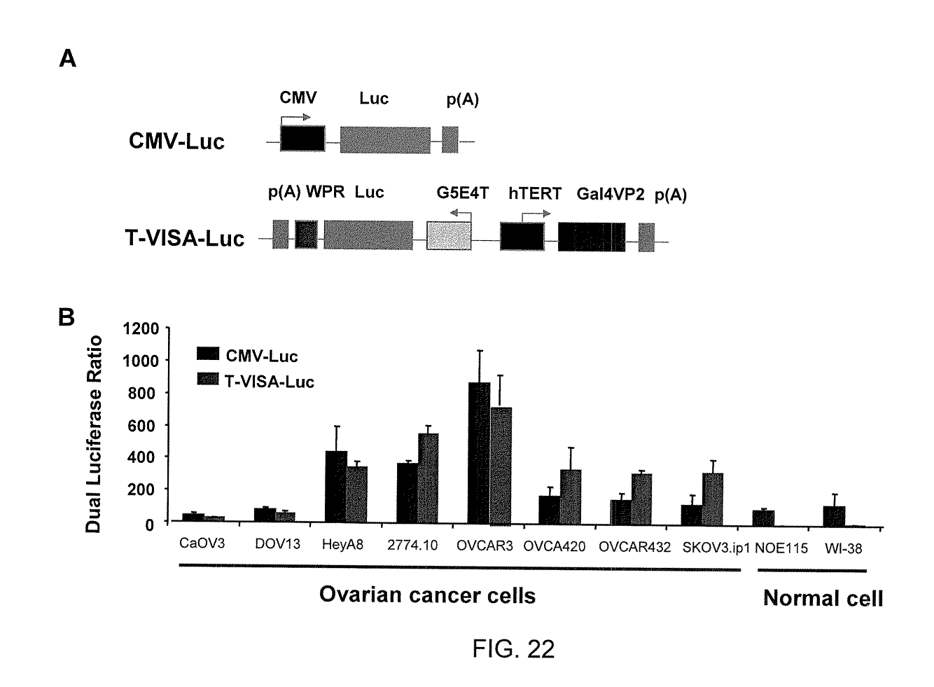

FIG. 22 shows that T-VISA is robust in ovarian cancer cell lines. In FIG. 22A, there is a schematic diagram of engineered hTERT-VISA constructs in the pGL3 backbone. In FIG. 22B, there are ovarian cancer cell lines, normal ovarian epithelia cells (NOE115) and fibroblasts (WI-38) that were transiently cotransfected with the indicated plasmid DNA and pRL-TK. Forty-eight hours later, dual luciferase ratio was measured and shown as RLU (ratio) normalized to the Renilla luciferase control. The data represent the mean of four independent experiments. Bar, SD.

### Example 20

### T-VISA Targets Transgene Expression in Ovarian Cancer Cells

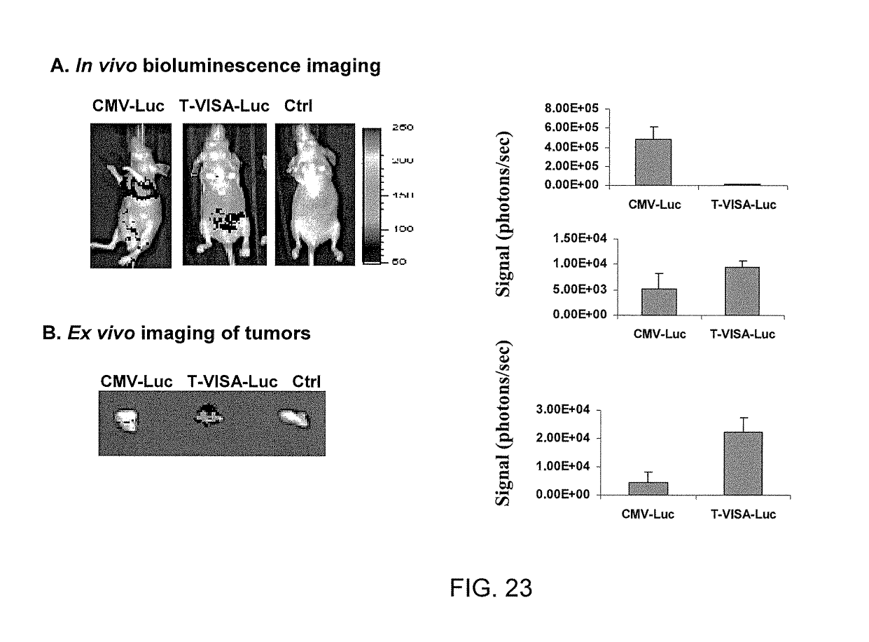

FIG. 23 shows that T-VISA transcriptionally targets transgene expression to ovarian cancer cells in vivo. Female nude mice bearing orthotopic HeyA8 tumors were given 50 μg of DNA in a DNA:liposome complex via the tail vein. Two days later, mice were anesthetized and subjected to in vivo imaging for 2 min at 10 min after intraperitoneal injection of d-luciferin (FIG. 23A). HeyA8 tumors of mice from A were subjected to ex vivo imaging (FIG. 23B). The photon signals were quantified by Xenogen's Living Imaging software (shown on the right). Bars, SD; n=3 per group. CMV-Luc, pGL3-CMV-Luc; T-VISA-Luc, pGL3-hTERT-VISA-Luc; Ctrl, pGL3-C-VISA.

### Example 21

### Cell Killing Activities of CMV-E1A and T-VISA-E1A in Ovarian Cancer Cells

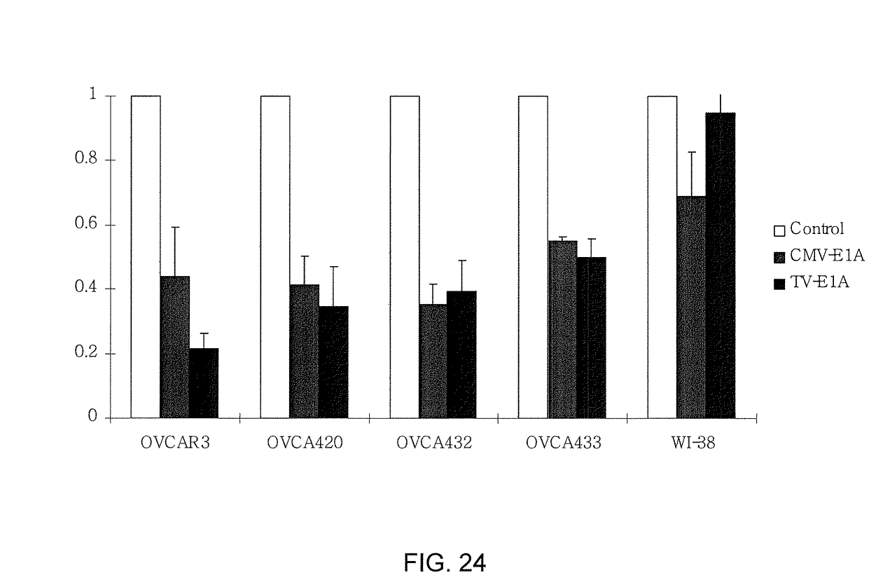

FIG. 24 demonstrates cell-killing activities of CMV-E1A and T-VISA-EIA in ovarian cancer cell lines and normal cells. A panel of ovarian cancer cell lines and normal fibroblasts were cotransfected with pUK21-T-VISA-E1A, pUK21-CMV-EIA, and negative control (pUK21-TV), plus 100 ng of pGL3-CMV-Luc. The signal was imaged with the IVIS system two days after transfection. The percentage of the signals as compared with the negative control (setting at 100%) was presented. The data represent the mean of three independent experiments. Bars, SD.

### REFERENCES

All patents and publications mentioned in the specification are indicative of the level of those skilled in the art to which the invention pertains. All patents and publications are herein incorporated by reference in their entirety to the same extent as if each individual publication was specifically and individually indicated to be incorporated by reference.

### Patents

U.S. Pat. No. 4,683,202
U.S. Pat. No. 4,797,368
U.S. Pat. No. 4,879,236
U.S. Pat. No. 5,139,941
U.S. Pat. No. 5,220,007
U.S. Pat. No. 5,284,760
U.S. Pat. No. 5,354,670
U.S. Pat. No. 5,366,878
U.S. Pat. No. 5,389,514
U.S. Pat. No. 5,635,377
U.S. Pat. No. 5,641,484
U.S. Pat. No. 5,670,488
U.S. Pat. No. 5,789,166
U.S. Pat. No. 5,798,208
U.S. Pat. No. 5,830,650
U.S. Pat. No. 5,871,986
U.S. Pat. No. 5,925,565
U.S. Pat. No. 5,928,906
U.S. Pat. No. 5,935,819

### Publications

Adachi, N., Nomoto, M., Kohno, K. & Koyama, H. (2000). Gene, 245, 49-57.
Anderson, L. M., Krotz, S., Weitzman, S. A., and Thimmapaya, B. (2000). Breast cancer-specific expression of the Candida albicans cytosine deaminase gene using a transcriptional targeting approach. Cancer Gene Ther 7, 845-852.
Barron, L. G., Szoka Jr., F. C. (1999). Non-viral vectors for gene therapy. Huang, L., Hung, M. C., Wagner, E. (ed.). Academic Press: San Diego, Calif., pp 229-266.
Bartke, T., Siegmund, D., Peters, N., Reichwein, M., Henkler, F., Scheurich, P., and Wajant, H. p53 upregulates cFLIP, inhibits transcription of NF-kappaB-regulated genes and induces caspase-8-independent cell death in DLD-1 cells, Oncogene. 20: 571-80., 2001.
Boyd, J. M., Gallo, G. J., Elangovan, B., Houghton, A. B., Malstrom, S., Avery, B. J., Ebb, R. G., Subramanian, T., Chittenden, T., Lutz, R. J., and et al. Bik, a novel death-inducing protein shares a distinct sequence motif with Bcl-2 family proteins and interacts with viral and cellular survival-promoting proteins, Oncogene. 11: 1921-S., 1995.
Chen, C. D., D. S. Welsbie, C. Tran, S. H. Baek, R. Chen, R. Vessella, M. G. Rosenfeld, and C. L. Sawyers, Molecular determinants of resistance to antiandrogen therapy. Nat Med, 2004. 10(1): p. 33-39.
Chen, J. S., J. C. Liu, L. Shen, K. M. Rau, H. P. Kuo, Y. M. Li, D. Shi, Y. C. Lee, K. J. Chang, and M. C. Hung, Cancer-specific activation of the survivin promoter and its potential use in gene therapy. Cancer Gene Ther, 2004.
Daniel, P. T., Pun, K. T., Ritschel, S., Sturm, I., Holler, J., Dorken, B., and Brown, R. Expression of the death gene Bik/Nbk promotes sensitivity to drug-induced apoptosis in corticosteroid-resistant T-cell lymphoma and prevents tumor growth in severe combined immunodeficient mice, Blood. 94: 1100-7., 1999.
Desagher, S., Osen-Sand, A., Montessuit, S., Magnenat, E., Vilbois, F., Hochmann, A., Joumot, L., Antonsson, B., and Martinou, J. C. Phosphorylation of bid by casein kinases I and II regulates its cleavage by caspase 8, Mol. Cell. 8: 601-11, 2001.
Emami, K. H. and Carey, M. (1992) A synergistic increase in potency of a multimerized VP16 transcriptional activation domain. EMBO J. 11:5005-5012.
Fuernkranz, H. A., Schwob, J. E., and Lucas, J. J. (1991) Differential issue localization of oviduct and erythroid transferrin receptors. Proc. Natl. Acad. Sci. USA 88:7505-7508.
Han, J., Sabbatini, P., and White, E. Induction of apoptosis by human Nbk/Bik, a BH3-containing protein that interacts with E1B 19K, Mol Cell Biol. 16: 5857-64., 1996.
Hochhauser, D., Stanway, C. A., Harris, A. L., Hickson, I. D. (1992) Cloning and characterization of the 5′ flanking region of the human topoisomerase II alpha gene. J. Biol. Chem. 267(26):18961-5.
Iyer, M., Wu, L., Carey, M., Wang, Y., Smallwood, A., and Gambhir, S. S. (2001). Two-step transcriptional amplification as a method for imaging reporter gene expression using weak promoters. Proc. Natl. Acad. Sci. 98:14595-14600.
Katabi, M. M., Chan, H. L., Karp, S. E., and Batist, G. (1999). Hexokinase type II: a novel tumor-specific promoter for gene-targeted therapy differentially expressed and regulated in human cancer cells. Hum Gene Ther 10, 155-164.
Klumpp, S, and Krieglstein, J. Serine/threonine protein phosphatases in apoptosis, Curr Opin Pharmacol. 2: 458-62., 2002.
Li, F., and Altieri, D. C. (1999) Biochem. J. 344, 305-311.
Li, S., Rizzo, M. A., Bhattacharya, S. & Huang, L. (1998). Gene Ther, 5, 930-7.
Li, Y. M., Wen, Y., Zhou, B. P., Kuo, H. P., Ding, Q. & Hung, M. C. (2003). Cancer Res, 63, 7630-3.
Lu, H., Zhang, Y., Roberts, D. D., Osborne, C. K., and Templeton, N. S. (2002). Enhanced gene expression in breast cancer cells in vitro and tumors in vivo. Mol Ther 6, 783-792.
Maeda, T., J, O. W., Matsubara, H., Asano, T., Ochiai, T., Sakiyama, S., and Tagawa, M. (2001). A minimum c-erbB-2 promoter-mediated expression of herpes simplex virus thymidine kinase gene confers selective cytotoxicity of human breast cancer cells to ganciclovir. Cancer Gene Ther 8, 890-896.
Mathai, J. P., Germain, M., Marcellus, R. C., and Shore, G. C. Induction and endoplasmic reticulum location of BIK/NBK in response to apoptotic signaling by E1A and p53, Oncogene. 21: 2534-44., 2002.
Menendez J A, Ropero S. Mehmi I, Atlas E, Colomer R, Lupu R. Overexpression and hyperactivity of breast cancer-associated fatty acid synthase (oncogenic antigen-519) is insensitive to normal arachidonic fatty acid-induced suppression in lipogenic tissues but it is selectively inhibited by tumoricidal alpha-linolenic and gamma-linolenic fatty acids: a novel mechanism by which dietary fat can alter mammary tumorigenesis. Int J Oncol. 2004 June; 24(6). 1369-83.
Mo, Y. Y., Ameiss, K. A., and Beck, W. T. (1998) Overexpression of human DNA topoisomerase II alpha by fusion to enhanced green fluorescent protein. Biotechn. 25(6):1052-7.
Nettelbeck, D. M., Jerome, V. and Muller, R. (2000) Gene therapy: designer promoters for tumour targeting. Trends Genet. 16:174-181.
Panaretakis, T., Pokrovskaja, K., Shoshan, M. C., and Grander, D. Activation of Bak, Bax, and BH3-only Proteins in the Apoptotic Response to Doxorubicin, J Biol Chem. 277: 44317-26., 2002.
Puthalakath, H. and Strasser, A. Keeping killers on a tight leash: transcriptional and post-translational control of the pro-apoptotic activity of BH3-only proteins, Cell Death Differ. 9: 505-12, 2002.
Qiao, J., Doubrovin, M., Sauter, B. V., Huang, Y., Guo, Z. S., Balatoni, J., Akhurst, T., Blasberg, R. G., Tjuvajev, J. G., Chen, S. H., and Woo, S. L. (2002). Tumor-specific transcriptional targeting of suicide gene therapy. Gene Ther 9, 168-175.
Sadowski I, Ma J, Triezenberg S, Ptashne M. GAL4-VP16 is an unusually potent transcriptional activator. Nature. 1988 Oct. 6; 335(6190):563-4.
Sato, M., Johnson, M., Zhang, L., Zhang, B., Le. K., Gambhir, S. S., Carey, M., Wu, L. (2003) Optimization of adenoviral vectors to direct highly amplified prostate-specific expression for imaging and gene therapy. Mol. Ther. 8(5):726-737.
Soini Y. Claudins 2, 3, 4, and 5 in Paget's disease and breast carcinoma. Hum Pathol. 2004 December; 35(12):1531-6.
Shindelman, J. E., Ortmeyer, a. E., and Sussman, H. H. (1981) Demonstration of the Transferrin Receptor in human breast cancer tissue. Potential marker for identifying dividing cells. Int. J. Cancer 27:329-334.
Shterman, N., Kupfer, B., and Moroz, C. (1991) Comparison of Transferrin Receptors, Iron Content and Isoferritin profile in normal and malignant human breast cell lines. Pathobiol. 59:19-25.
Templeton, N. S., Lasic, D. D., Frederik, P. M., Strey, H. H., Roberts, D. D. & Pavlakis, G. N. (1997). Nat Biotechnol, 15, 647-52.
Theodorakis, P., Lomonosova, E., and Chinnadurai, G. Critical requirement of BAX for manifestation of apoptosis induced by multiple stimuli in human epithelial cancer cells, Cancer Res. 62: 3373-6., 2002.
Verma, S., Zhao, L., and Chinnadurai, G. Phosphorylation of the Pro-Apoptotic Protein BIK: Mapping of Phosphorylation sites and Effect on Apoptosis, J Biol Chem. 17: 17, 2000.
Visakorpi, T., E. Hyytinen, P. Koivisto, M. Tanner, R. Keinanen, C. Palmberg, A. Palotie, T. Tammela, J. Isola, and O. P. Kallioniemi, In vivo amplification of the androgen receptor gene and progression of human prostate cancer. Nat Genet, 1995. 9(4): p. 401-6.
Wang, H. G., Pathan, N., Ethell, I. M., Krajewski, S., Yamaguchi, Y., Shibasaki, F., McKeon, F., Bobo, T., Franke, T. F., and Reed, J. C. Ca2+-induced apoptosis through calcineurin dephosphorylation of BAD, Science. 284: 339-43., 1999.
Wang Y Y, Kuhajda F P, Li J, Finch T T, Cheng P, Koh C, Li T, Sokoll Lj, Chan D W. Fatty acid synthase as a tumor marker: its extracellular expression in human breast cancer. J Exp Ther Oncol. 2004 July; 4(2): 101-10.
Xie X, Luo Z, Slawin K M, Spencer D M. The EZC-prostate model: noninvasive prostate imaging in living mice. Mol Endoclinol. 2004 March; 18(3):722-32.
Xie, X., X. Zhao, Y. Liu, J. Zhang, R. J. Matusik, K. M. Slawin, and D. M. Spencer, Adenovirus-mediated tissue-targeted expression of a caspase-9-based artificial death switch for the treatment of prostate cancer. Cancer Res, 2001. 61(18): p. 6795-804.
Zhang, L., Adams, J. Y., Billick, E., Ilagan, R., Iyer, M., Le, K., Smallwood, A., Gambhir, S. S., Carey, M., Wu, L. (2002) Molecular engineering of a two-step transcription amplification (TSTA) system for transgene delivery in prostate cancer. Mol. Ther. 5(3): 223-232.
Zhang, J., T. Z. Thomas, S. Kasper, and R. J. Matusik, A small composite probasin promoter confers high levels of prostate-specific gene expression through regulation by androgens and glucocorticoids in vitro and in vivo. Endocrinology, 2000. 141(12): p. 4698-710.
Zou, Y., Peng, H., Zhou, B., Wen, Y., Wang, S. C., Tsai, E. M., and Hung, M. C. Systemic tumor suppression by the proapoptotic gene bik, Cancer Res. 62: 8-12., 2002.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

We claim:

1. A polynucleotide construct comprising a breast cancer-specific control sequence and one or both of the following:
a post-transcriptional regulatory sequence; and
a two-step transcriptional amplification (TSTA) sequence, said TSTA sequence including a DNA binding domain and an activation domain.

2. The construct of claim 1, wherein said breast cancer-specific control sequence comprises fatty acid synthase control sequence, claudin 4 control sequence, or both.

3. The construct of claim 1, further comprising an enhancer.

4. The construct of claim 3, wherein the enhancer comprises cytomegalovirus (CMV) enhancer, Glyceraldehyde-3-phosphate dehydrogenase promoter (GAPDH), or the m-actin promoter.

5. The construct of claim 1, wherein the post-transcriptional regulatory sequence is woodchuck hepatitis virus post-transcriptional regulatory element (WPRE).

6. The construct of claim 1, wherein the DNA binding domain is Gal1, Gal4, or LexA.

7. The construct of claim 1, wherein the activation domain is VP2 or VP16.

8. The construct of claim 1, wherein the TSTA sequence is GAL4-VP2 or GAL4-VP16.

9. The construct of claim 1, wherein said control sequence is operably linked to a polynucleotide encoding a therapeutic gene product.

10. The construct of claim 9, wherein the therapeutic gene product comprises an inhibitor of cell proliferation, a regulator of programmed cell death, or a tumor suppressor.

11. The construct of claim 9, wherein the therapeutic gene product is a mutant Bik or E1A.

12. The construct of claim 11, wherein the mutant Bik comprises an amino acid substitution at threonine 33, serine 35, or both.

13. The construct of claim 1, further defined as being comprised in a liposome.

14. A polynucleotide construct comprising an ovarian cancer-specific control sequence and one or both of the following:
a post-transcriptional regulatory sequence; and
a two-step transcriptional amplification (TSTA) sequence, said TSTA sequence including a DNA binding domain and an activation domain.

15. The construct of claim 14, wherein said ovarian cancer-specific control sequence comprises hTERT control sequence, survivin control sequence, or both.

16. The construct of claim 14, further comprising an enhancer.

17. The construct of claim 16, wherein the enhancer comprises cytomegalovirus (CMV) enhancer, Glyceraldehyde-3-phosphate dehydrogenase promoter (GAPDH), or the M-actin promoter.

18. The construct of claim 14, wherein the post-transcriptional regulatory sequence is woodchuck hepatitis virus post-transcriptional regulatory element (WPRE).

19. The construct of claim 14, wherein the DNA binding domain is Gal1, Gal4, or LexA.

20. The construct of claim 14, wherein the activation domain is VP2 or VP16.

21. The construct of claim 14, wherein the TSTA sequence is GAL4-VP2 or GAL4-VP16.

22. The construct of claim 14, wherein said control sequence is operably linked to a polynucleotide encoding a therapeutic gene product.

23. The construct of claim 22, wherein the therapeutic gene product comprises an inhibitor of cell proliferation, a regulator of programmed cell death, or a tumor suppressor.

24. The construct of claim 22, wherein the therapeutic gene product is a mutant Bik or E1A.

25. The construct of claim 24, wherein the mutant Bik comprises an amino acid substitution at threonine 33, serine 35, or both.

26. The construct of claim 14, further defined as being comprised in a liposome.

27. A method of inhibiting breast cancer cell proliferation, comprising contacting a breast cancer cell with an effective amount of a polynucleotide construct that comprises the construct of claim 1, said construct operably linked to a polynucleotide encoding a gene product effective to inhibit the cell proliferation.

28. A method of inhibiting ovarian cancer cell proliferation, comprising contacting an ovarian cancer cell with an effective amount of a polynucleotide construct that comprises the construct of claim 14, said construct operably linked to a polynucleotide encoding a gene product effective to inhibit the cell proliferation.

29. A method of treating breast cancer in an individual having the cancer, comprising contacting at least one breast cancer cell of the individual with a therapeutically effective amount of the construct of claim 1, said construct operably linked to a polynucleotide encoding a gene product effective to treat breast cancer.

30. A method of treating ovarian cancer in an individual having the cancer, comprising contacting at least one ovarian cancer cell of the individual with a therapeutically effective amount of the construct of claim 1, said construct operably linked to a polynucleotide encoding a gene product effective to treat ovarian cancer.
